(19) United States
(12) Patent Application Publication
(10) Pub. No.: US 2018/0028670 A1
(43) Pub. Date: Feb. 1, 2018

(54) TAMPER RESISTANT DOSAGE FORM COMPRISING AN ANIONIC POLYSACCHARIDE

(71) Applicant: Grünenthal GMBH, Aachen (DE)

(72) Inventors: Klaus WENING, Köln (DE); Carmen Stomberg, Korschenbroich (DE)

(73) Assignee: Grünenthal GMBH, Aachen (DE)

(21) Appl. No.: 15/665,549

(22) Filed: Aug. 1, 2017

(30) Foreign Application Priority Data
Aug. 1, 2016 (EP) 16182124.4

(51) Int. Cl. A61K 47/38; A61K 31/485; A61K 31/137; A61K 47/10

(52) U.S. Cl. CPC A61K 47/38; A61K 47/10; A61K 31/485; A61K 31/137

(57) ABSTRACT

A pharmaceutical dosage form having a breaking strength of at least 300 N, said dosage form comprising:
an opioid (A) selected from Oxymorphone, Oxycodone, Tapentadol, Hydromorphone, Hydrocodone, Morphine, and physiologically acceptable salts thereof; wherein the weight content of the opioid (A) is from 5.0 to 35 wt.-%;
an anionic polysaccharide (B) selected from croscarmellose, carmellose, crosslinked carboxymethyl starch, carboxymethyl starch, and physiologically acceptable salts thereof; wherein the weight content of the anionic polysaccharide (B) is within from 5.0 to 35 wt.-%; and
a polyalkylene oxide (C) having a weight average molecular weight of at least 200,000 g/mol; wherein the weight content of the polyalkylene oxide (C) is from 20 to 80 wt.-%;
   wherein all wt.-%'s are based on a total weight of the dosage form, and the opioid (A) is present in a controlled-release matrix comprising the anionic polysaccharide (B) and the polyalkylene oxide (C).

This application claims priority of EP 16182124.4, filed on Aug. 1, 2016, the entire contents of which are hereby incorporated herein by reference.

The invention relates to a pharmaceutical dosage form having a breaking strength of at least 300 N, said dosage form comprising
an opioid (A) selected from the group consisting of Oxymorphone, Oxycodone, Tapentadol, Hydromorphone, Hydrocodone, Morphine, and the physiologically acceptable salts thereof; wherein the weight content of the opioid (A) is within the range of from 5.0 to 35 wt.-%, based on the total weight of the pharmaceutical dosage form;
an anionic polysaccharide (B) selected from the group consisting of croscarmellose, carmellose, crosslinked carboxymethyl starch, carboxymethyl starch, and the physiologically acceptable salts thereof; wherein the weight content of the anionic polysaccharide (B) is within the range of from 5.0 to 35 wt.-%, based on the total weight of the pharmaceutical dosage form; and
a polyalkylene oxide (C) having a weight average molecular weight of at least 200,000 g/mol; wherein the weight content of the polyalkylene oxide (C) is within the range of from 20 to 80 wt.-%, based on the total weight of the pharmaceutical dosage form;
   wherein the opioid (A) is present in a controlled-release matrix comprising the anionic polysaccharide (B) and the polyalkylene oxide (C).

Many pharmacologically active ingredients have a potential of being abused and thus, are advantageously provided in form of tamper resistant pharmaceutical dosage forms. Prominent examples of such pharmacologically active ingredients are opioids. It is known that abusers crush conventional tablets, which contain opioids, to defeat the time-release “microencapsulation” and then ingest the resulting powder orally, intra-nasally, rectally, or by injection. Injection typically requires that the active ingredient is extracted from the powder. Typical solvents employed for that purpose are water and ethanol.

Various concepts for the avoidance of drug abuse have been developed. One concept relies on the mechanical properties of the pharmaceutical dosage forms, particularly an increased breaking strength (resistance to crushing). The major advantage of such pharmaceutical dosage forms is that comminuting, particularly pulverization, by conventional means, such as grinding in a mortar or fracturing by means of a hammer, is impossible or at least substantially impeded. Such pharmaceutical dosage forms are useful for avoiding drug abuse of the pharmacologically active ingredient contained therein, as they may not be powdered by conventional means and thus, cannot be administered in powdered from, e.g. nasally. The mechanical properties, particularly the high breaking strength of these pharmaceutical dosage forms renders them tamper resistant. In the context of such tamper resistant pharmaceutical dosage forms it can be referred to, e.g., US 2005/031546, WO 2005/016313, WO 2005/016314, WO 2005/063214, WO 2005/102286, WO 2006/002883, WO 2006/002884, WO 2006/002886, WO 2006/082097, WO 2006/082099, WO 2008/107149, and WO 2009/092601.

Another concept for avoiding drug abuse aims at preventing solvent extraction of the active ingredient from the dosage form. If a pharmaceutical dosage form provides resistance against solvent extraction, e.g. in water or ethanol, it is particularly more difficult to transform the active ingredient into a form suitable for parenteral abuse, e.g. by intravenous injection.

However, resistance against solvent extraction is difficult to achieve.

WO 2007/085024 discloses a dosage form and method for the delivery of drugs, particularly drugs of abuse, characterized by resistance to solvent extraction, tampering, crushing, or grinding, and providing an initial burst of release of drug followed by a prolonged period of controllable drug release.

WO 2012/028317 relates to a pharmaceutical dosage form exhibiting a breaking strength of at least 500 N, said dosage form containing a pharmacologically opioid (A); an anionic polysaccharide (B) obtainable by introducing anionic functional groups, in protonated form or a physiologically acceptable salt thereof, into a polysaccharide; and a polyalkylene oxide (C); wherein the pharmacologically opioid (A) is present in a controlled-release matrix comprising the anionic polysaccharide (B) and the polyalkylene oxide (C).

WO 2014/191397 relates to a tamper-resistant pharmaceutical dosage form comprising one or more particles, wherein each of said one or more particles comprises a pharmacologically active ingredient and a physiologically acceptable polymer; has a breaking strength of at least 300 N; has a weight of at least 2 mg; and optionally, comprises a film-coating; wherein the total weight of the pharmaceutical dosage form is greater than the total weight of said one or more particles.

There is a demand pharmaceutical dosage forms that contain opioids and that provide tamper resistance not only in terms of resistance against crushing but also in terms of resistance against solvent extraction.

It is an object of the invention to provide pharmaceutical dosage forms having advantages compared to pharmaceutical dosage forms of the prior art.

This object has been achieved by the subject-matter of the patent claims.

It has been surprisingly found that anionic polysaccharides such as croscarmellose, carmellose, crosslinked carboxymethyl starch, carboxymethyl starch, and the physiologically acceptable salts thereof are capable of providing pharmaceutical dosage forms with resistance against solvent extraction.

Further, it has been surprisingly found that based upon a combination of such anionic polysaccharides with polyalkylene oxides tamper-resistant pharmaceutical dosage forms can be provided that have an increased breaking strength and additionally an improved resistance against solvent extraction.

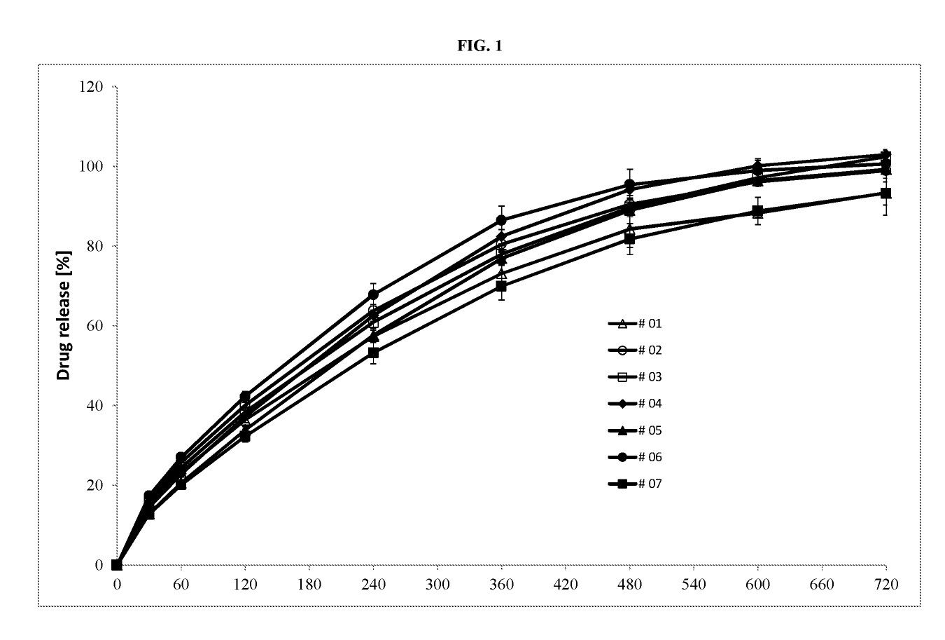

FIG. 1 compares the in vitro release profile of pharmaceutical dosage forms according to the invention with comparative pharmaceutical dosage forms in either case comprising Hydrocodone.

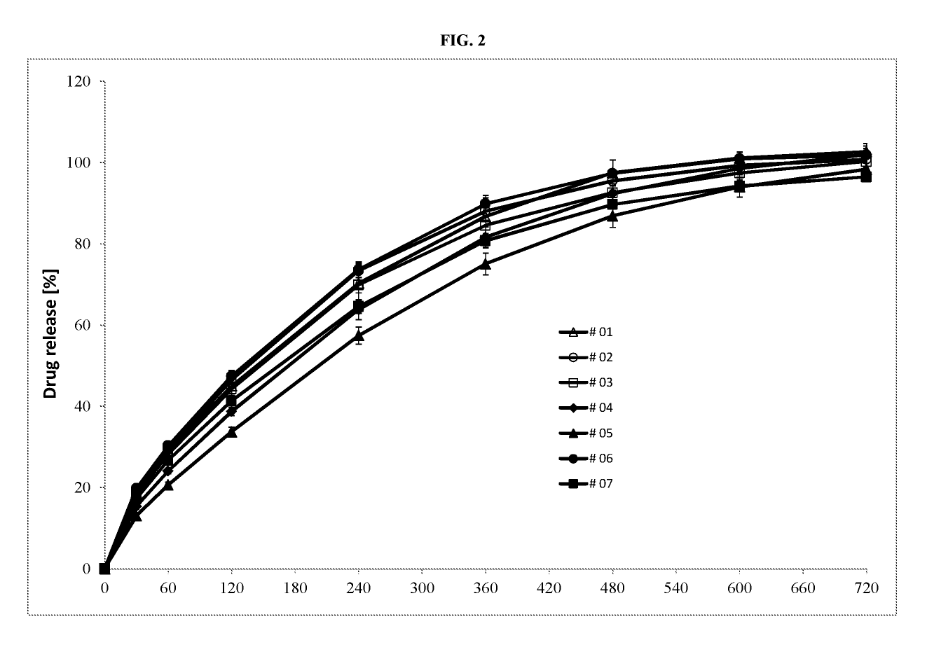

FIG. 2 compares the in vitro release profile of pharmaceutical dosage forms according to the invention with comparative pharmaceutical dosage forms in either case comprising Oxymorphone.

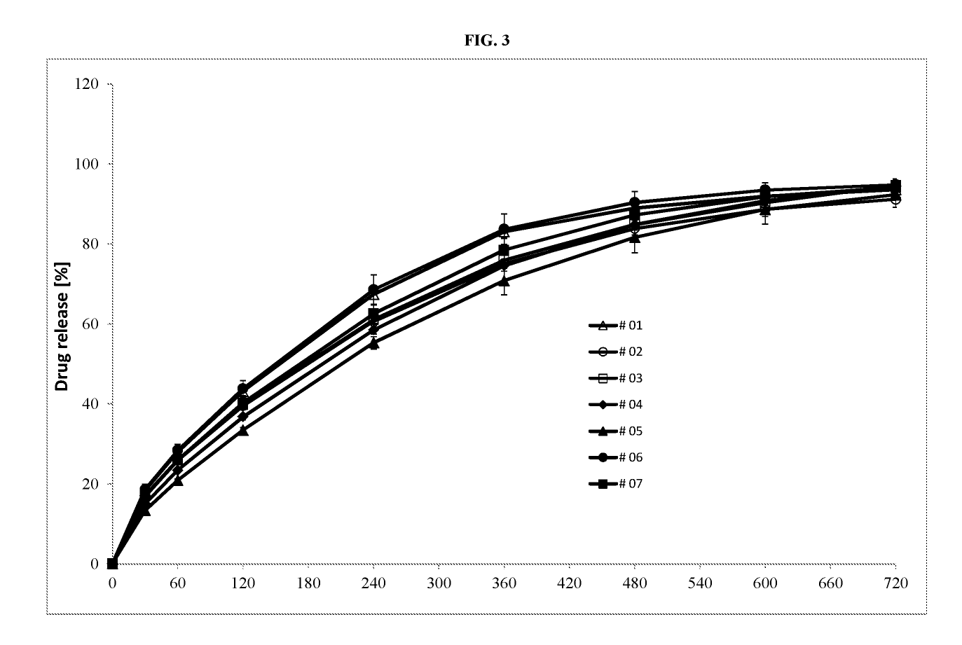

FIG. 3 compares the in vitro release profile of pharmaceutical dosage forms according to the invention with comparative pharmaceutical dosage forms in either case comprising Oxycodone.

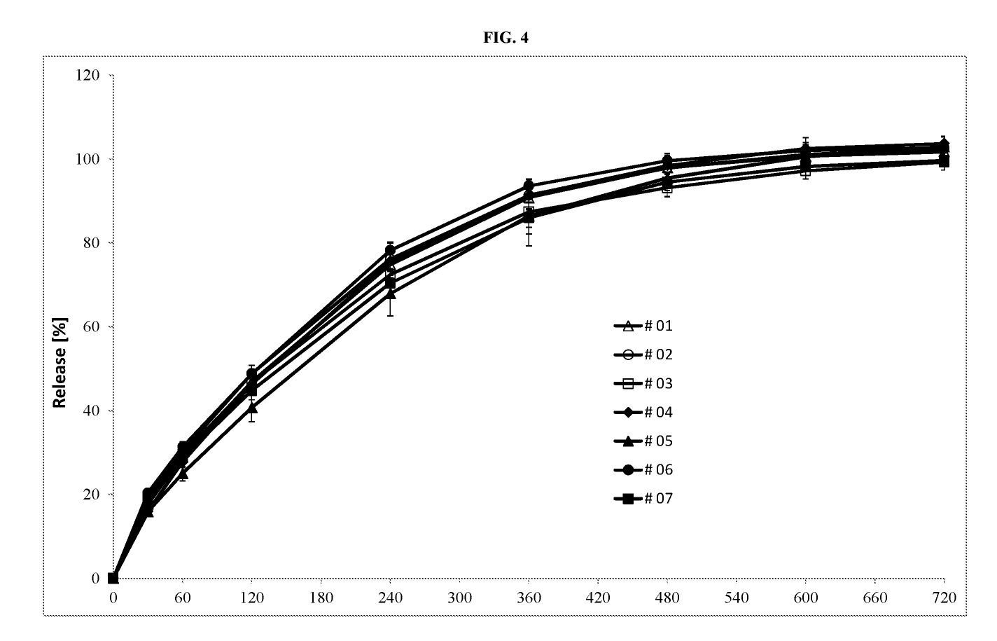

FIG. 4 compares the in vitro release profile of pharmaceutical dosage forms according to the invention with comparative pharmaceutical dosage forms in either case comprising Tapentadol.

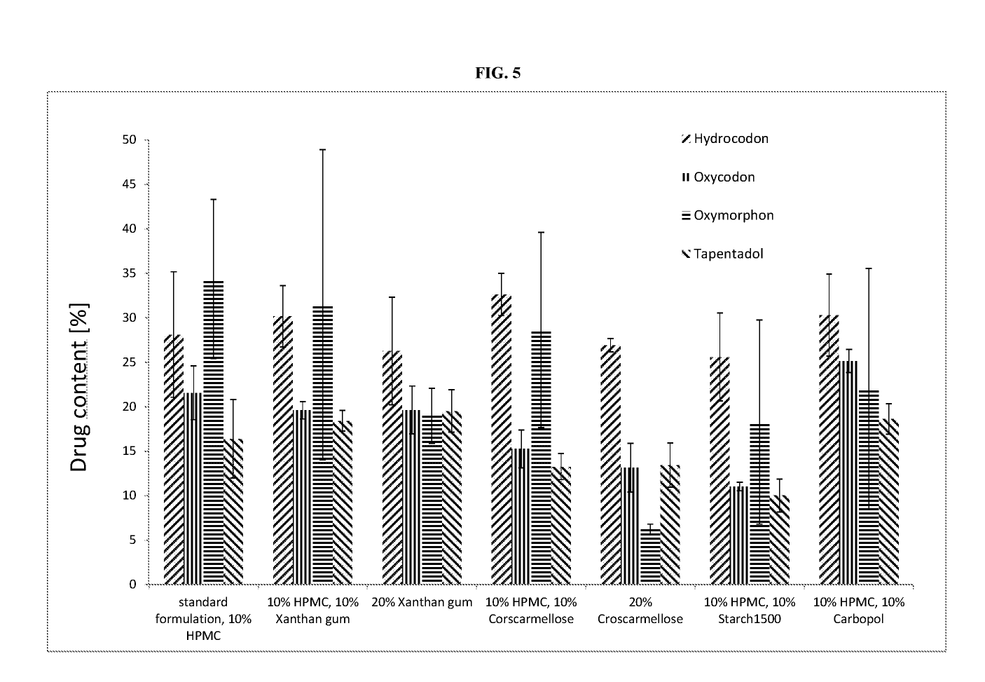

FIG. 5 shows the results of an extraction test with 5 ml water, boiled for 5 min.

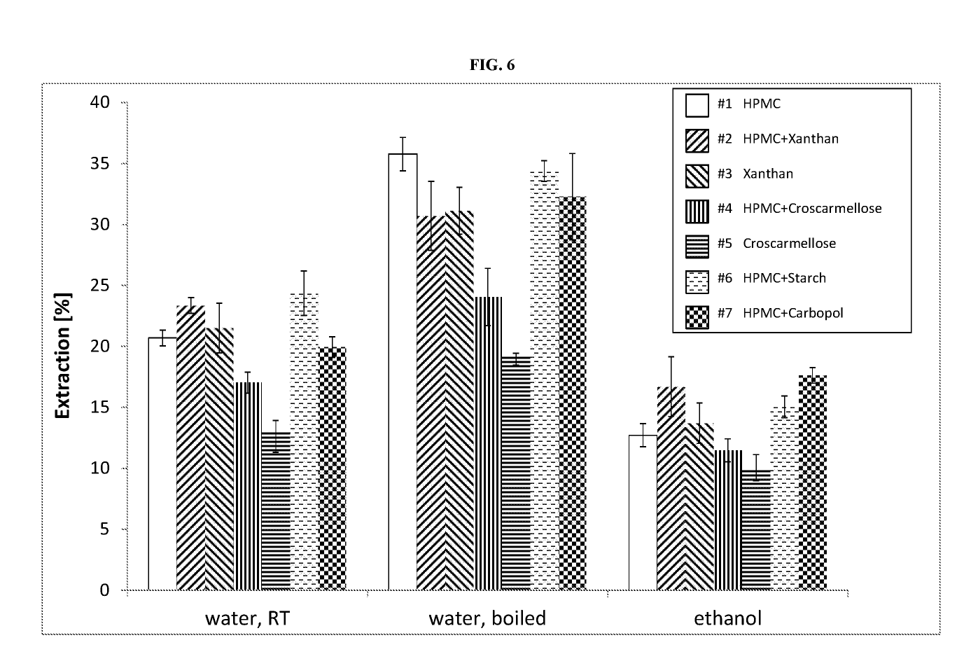

FIG. 6 shows the results of an extraction test for Hydrocodone with 30 ml medium, 30 min.

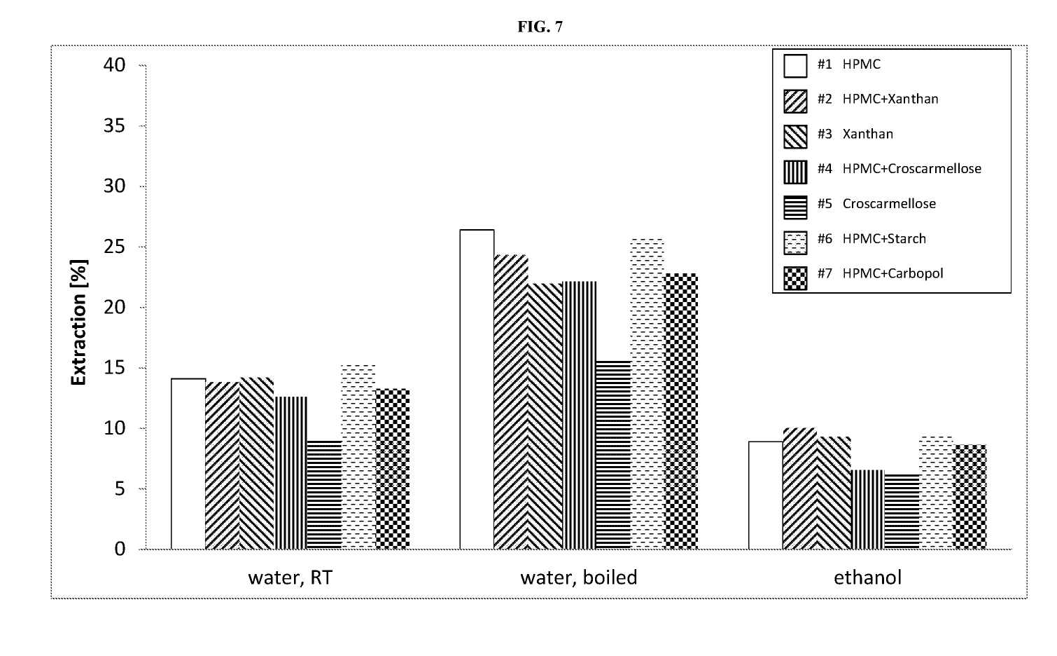

FIG. 7 shows the results of an extraction test for Oxymorphone with 30 ml medium, 30 min.

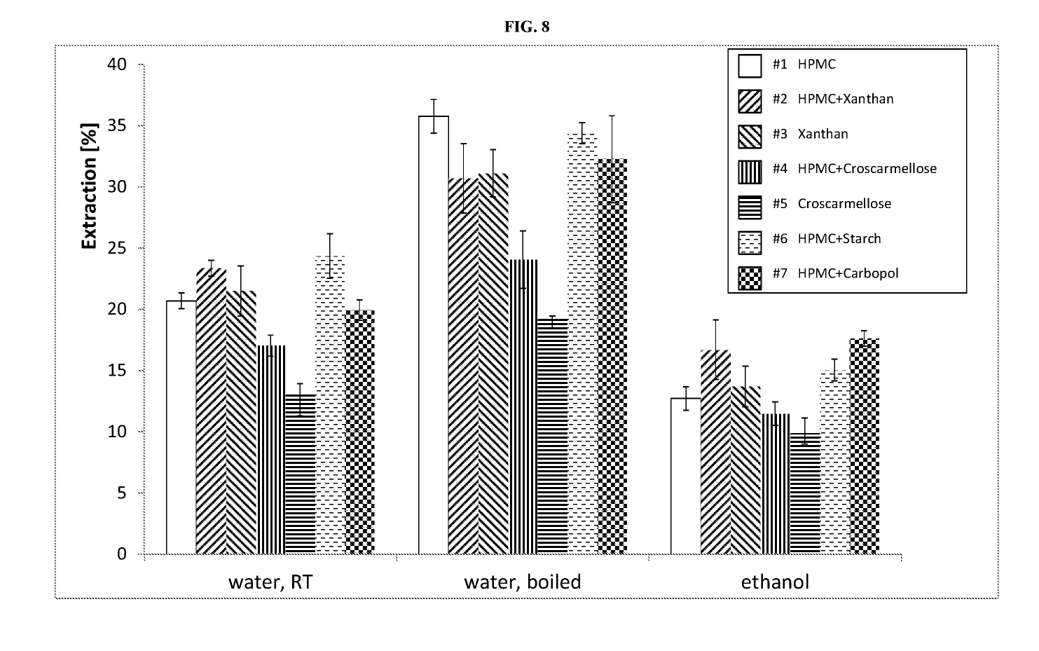

FIG. 8 shows the results of an extraction test for Oxycodone with 30 ml medium, 30 min.

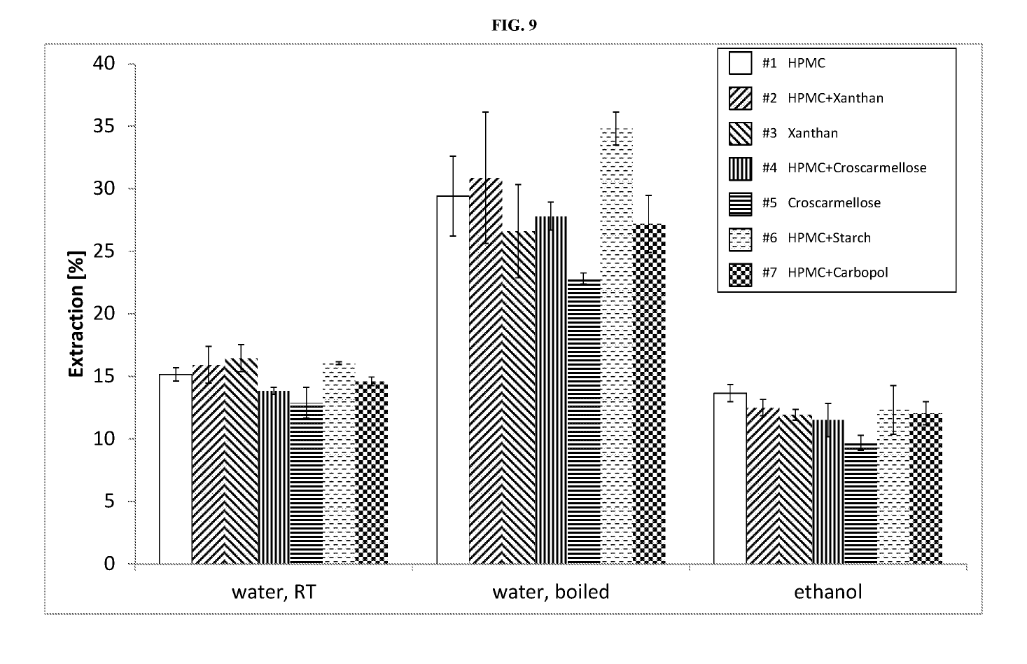

FIG. 9 shows the results of an extraction test for Tapentadol with 30 ml medium, 30 min.

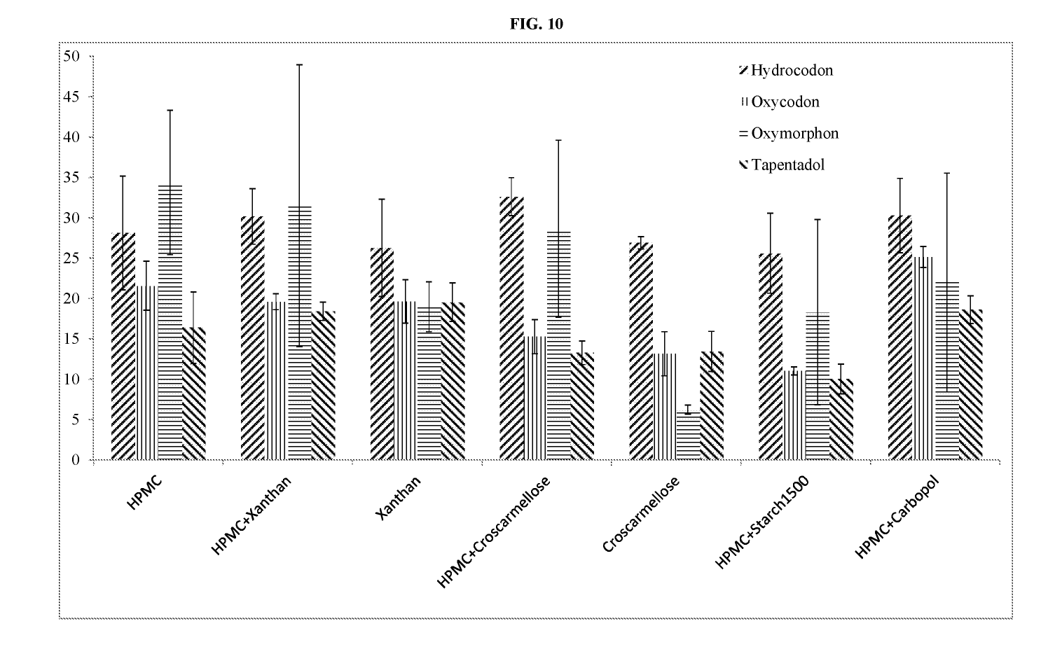

FIG. 10 compares the results of an extraction test for Hydrocodone, Oxymorphone, Oxycodone and Tapentadol.

A first aspect of the invention relates to a pharmaceutical dosage form having a breaking strength of at least 300 N, preferably of at least 500 N, said dosage form comprising
an opioid (A) selected from the group consisting of Oxymorphone, Oxycodone, Tapentadol, Hydromorphone, Hydrocodone, Morphine, and the physiologically acceptable salts thereof; wherein the weight content of the opioid (A) is preferably within the range of from 5.0 to 35 wt.-%, based on the total weight of the pharmaceutical dosage form;
an anionic polysaccharide (B) selected from the group consisting of croscarmellose, carmellose, crosslinked carboxymethyl starch, carboxymethyl starch, and the physiologically acceptable salts thereof; wherein the weight content of the anionic polysaccharide (B) is preferably within the range of from 5.0 to 35 wt.-%, based on the total weight of the pharmaceutical dosage form; and
a polyalkylene oxide (C) having a weight average molecular weight of at least 200,000 g/mol; wherein the weight content of the polyalkylene oxide (C) is preferably within the range of from 20 to 80 wt.-%, based on the total weight of the pharmaceutical dosage form; wherein the opioid (A) is present in a controlled-release matrix comprising the anionic polysaccharide (B) and the polyalkylene oxide (C).

For the purpose of the description, unless expressly stated otherwise, all percentages are weight percent (wt.-%).

For the purpose of the description, unless expressly stated otherwise, all values with regard to the content of opioid (e.g. in mg or in wt.-%) are expressed as weight equivalents with regard to the free base of the opioid.

The pharmaceutical dosage form according to the invention comprises an opioid (A) selected from the group consisting of Oxymorphone, Oxycodone, Tapentadol, Hydromorphone, Hydrocodone, Morphine, and the physiologically acceptable salts thereof. Preferably, the opioid (A) is Oxymorphone or a physiologically acceptable salt thereof.

In a preferred embodiment, the pharmaceutical dosage form contains opioid (A) as the sole pharmacologically active ingredient. In another preferred embodiment, the pharmaceutical dosage form contains a combination of opioid (A) with another pharmacologically active ingredient.

The opioid (A) may be present in form of a physiologically acceptable salt, e.g. physiologically acceptable acid addition salt. Preferred salts include but are not limited to bitartrates and hydrochlorides.

Physiologically acceptable salts comprise the acid addition salt forms which can conveniently be obtained by treating the base form of the opioid (A) with appropriate organic and inorganic acids. The salt also comprises the hydrates and solvent addition forms which the opioids (A) are able to form. Examples of such forms are e.g. hydrates, alcoholates and the like.

The opioid (A) is present in the dosage form in a therapeutically effective amount. The amount that constitutes a therapeutically effective amount varies according to the active ingredients being used, the condition being treated, the severity of said condition, the patient being treated, and whether the dosage form is designed for an immediate or retarded release.

The weight content of the opioid (A) is within the range of from 5.0 to 35 wt.-%, based on the total weight of the pharmaceutical dosage form. Preferably, the weight content of the opioid (A) is within the range of 20±10 wt.-%, based on the total weight of the dosage form.

Preferably, the weight content of the opioid (A) is within the range of 19±15 wt.-%, more preferably 19±13 wt.-%, still more preferably 19±11 wt.-%, yet more preferably 19±9 wt.-%, even more preferably 19±7 wt.-%, and most preferably 19±5 wt.-%, based on the total weight of the dosage form.

The absolute dose of the opioid (A) in the pharmaceutical dosage form is not limited. The dose of the opioid (A) which is adapted for administration preferably is in the range of 0.1 mg to 500 mg, more preferably in the range of 1.0 mg to 400 mg, even more preferably in the range of 5.0 mg to 300 mg, and most preferably in the range of 10 mg to 250 mg. In a preferred embodiment, the total amount of the opioid (A) that is contained in the pharmaceutical dosage form is within the range of from 0.01 to 200 mg, more preferably 0.1 to 190 mg, still more preferably 1.0 to 180 mg, yet more preferably 1.5 to 160 mg, most preferably 2.0 to 100 mg and in particular 2.5 to 80 mg.

In a preferred embodiment, the opioid (A) is contained in the pharmaceutical dosage form in an amount of 7.5±5 mg, 10±5 mg, 20±5 mg, 30±5 mg, 40±5 mg, 50±5 mg, 60±5 mg, 70±5 mg, 80±5 mg, 90±5 mg, 100±5 mg, 110±5 mg, 120±5 mg, 130±5, 140±5 mg, 150±5 mg, or 160±5 mg. In another preferred embodiment, the opioid (A) is contained in the pharmaceutical dosage form in an amount of 5±2.5 mg, 7.5±2.5 mg, 10±2.5 mg, 15±2.5 mg, 20±2.5 mg, 25±2.5 mg, 30±2.5 mg, 35±2.5 mg, 40±2.5 mg, 45±2.5 mg, 50±2.5 mg, 55±2.5 mg, 60±2.5 mg, 65±2.5 mg, 70±2.5 mg, 75±2.5 mg, 80±2.5 mg, 85±2.5 mg, 90±2.5 mg, 95±2.5 mg, 100±2.5 mg, 105±2.5 mg, 110±2.5 mg, 115±2.5 mg, 120±2.5 mg, 125±2.5 mg, 130±2.5 mg, 135±2.5 mg, 140±2.5 mg, 145±2.5 mg, 150±2.5 mg, 155±2.5 mg, or 160±2.5 mg.

In a particularly preferred embodiment, opioid (A) is Hydrocodone, preferably its bitartrate salt, and the pharmaceutical dosage form is adapted for administration twice daily. In this embodiment, opioid (A) is preferably contained in the pharmaceutical dosage form in an amount of from 5 to 200 mg. In another particularly preferred embodiment, opioid (A) is Hydrocodone, preferably its bitartrate salt, and the pharmaceutical dosage form is adapted for administration once daily. In this embodiment, opioid (A) is preferably contained in the pharmaceutical dosage form in an amount of from 10 to 400 mg.

In another particularly preferred embodiment, opioid (A) is Oxymorphone, preferably its HCl salt, and the pharmaceutical dosage form is adapted for administration twice daily. In this embodiment, opioid (A) is preferably contained in the pharmaceutical dosage form in an amount of from 5 to 40 mg. In another particularly preferred embodiment, opioid (A) is Oxymorphone, preferably its HCl, and the pharmaceutical dosage form is adapted for administration once daily. In this embodiment, opioid (A) is preferably contained in the pharmaceutical dosage form in an amount of from 10 to 80 mg.

In still another particularly preferred embodiment opioid (A) is Oxycodone, preferably its HCl salt, and the pharmaceutical dosage form is adapted for administration twice daily. In this embodiment, opioid (A) is preferably contained in the pharmaceutical dosage form in an amount of from 5 to 80 mg. In another particularly preferred embodiment, opioid (A) is Oxycodone, preferably its HCl, and the pharmaceutical dosage form is adapted for administration once daily. In this embodiment, opioid (A) is preferably contained in the pharmaceutical dosage form in an amount of from 10 to 320 mg.

In yet another particularly preferred embodiment, opioid (A) is Tapentadol, preferably its HCl salt, and the pharmaceutical dosage form is adapted for administration once daily or twice daily. In this embodiment, opioid (A) is preferably contained in the pharmaceutical dosage form in an amount of from 25 to 250 mg.

The opioid (A) is present in a controlled-release matrix comprising the anionic polysaccharide (B) and the polyalkylene oxide (C).

The pharmaceutical dosage form according to the invention comprises an anionic polysaccharide (B) selected from the group consisting of croscarmellose, carmellose, crosslinked carboxymethyl starch, carboxymethyl starch, and the physiologically acceptable salts thereof. The opioid (A) is embedded into a controlled-release matrix comprising said anionic polysaccharide (B).

The anionic polysaccharide (B) may be linear or branched (carmellose or carboxymethyl starch) and/or cross-linked (croscarmellose or crosslinked carboxymethyl starch).

Preferably, at least some of the carboxylic groups contained in the anionic polysaccharide (B) are present in neutralized form, i.e. they are not present in their protonated forms, but are salts with salt-forming cations instead. Suitable salt-forming cations include alkali metal, ammonium, substituted ammonium and amines. More preferably, at least some of the anionic functional groups, e.g. carboxylate and/or sulfonate anions, are salts of sodium or potassium cations.

In a preferred embodiment, the anionic polysaccharide (B) is carmellose or a physiologically acceptable salt thereof. Preferably, the anionic polysaccharide (B) is carmellose in accordance with monograph E-52 Carmellose of USP, preferably in the version of 2016, or a salt thereof, preferably carmellose calcium in accordance with monograph E-07 Carmellose Calcium of USP, preferably in the version of 2016; or carmellose sodium in accordance with monograph E-08 Carmellose Sodium of USP, preferably in the version of 2016.

In another preferred embodiment, the anionic polysaccharide (B) is starch glycolate or a physiologically acceptable salt thereof. Preferably, the anionic polysaccharide (B) is sodium starch glycolate in accordance with monograph E-39 Sodium Starch Glycolate of USP, preferably in the version of 2016.

In a particularly preferred embodiment, the anionic polysaccharide (B) is croscarmellose or a physiologically acceptable salt thereof. Preferably, the anionic polysaccharide (B) is croscarmellose sodium. Preferably, the croscarmellose sodium is in accordance with monograph E-09 Croscarmellose Sodium of USP, preferably in the version of 2016.

Croscarmellose sodium is an internally cross-linked sodium carboxymethylcellulose typically used as a superdisintegrant in pharmaceutical formulations. The cross-linking reduces water solubility while still allowing the material to swell and absorb many times its weight in water. Its purpose in most tablets—including dietary supplements—is to assist the tablet in disintegrating in the gastrointestinal tract promptly. Croscarmellose can be made by first soaking crude cellulose in sodium hydroxide, and then reacting the cellulose with sodium monochloroacetate to form sodium carboxymethylcellulose. Excess sodium monochloroacetate slowly hydrolyzes to glycolic acid and the glycolic acid catalyzes the cross-linkage to form croscarmellose sodium. Chemically, croscarmellose sodium is the sodium salt of a cross-linked, partly O-(carboxymethylated) cellulose.

The weight content of the anionic polysaccharide (B) is within the range of from 5.0 to 35 wt.-%, based on the total weight of the pharmaceutical dosage form.

In a preferred embodiment, especially when the pharmaceutical dosage form does not contain a nonionic polysaccharide, the weight content of the anionic polysaccharide (B) is within the range of 20±15 wt.-%, more preferably 20±13 wt.-%, still more preferably 20±11 wt.-%, yet more preferably 20±9 wt.-%, even more preferably 20±7 wt.-%, and most preferably 20±5 wt.-%, based on the total weight of the dosage form.

In another preferred embodiment, especially when the pharmaceutical dosage form additionally contains a nonionic polysaccharide, preferably HPMC, the weight content of the anionic polysaccharide (B) is within the range of 10±9 wt.-%, more preferably 10±8 wt.-%, still more preferably 10±7 wt.-%, yet more preferably 10±6 wt.-%, even more preferably 10±5 wt.-%, and most preferably 10±4 wt.-%, based on the total weight of the dosage form.

In a preferred embodiment, anionic polysaccharide (B) is homogeneously distributed in the pharmaceutical dosage form according to the invention. Preferably, the opioid (A) and anionic polysaccharide (B) are intimately homogeneously distributed in the pharmaceutical dosage form so that the pharmaceutical dosage form does not contain any segments where either opioid (A) is present in the absence of anionic polysaccharide (B) or where anionic polysaccharide (B) is present in the absence of opioid (A).

When the pharmaceutical dosage form is film coated, the anionic polysaccharide (B) is preferably homogeneously distributed in the core of the pharmaceutical dosage form, i.e. the film coating preferably does not contain anionic polysaccharide (B). Nonetheless, the film coating as such may of course contain one or more polymers, which however, preferably differ from the anionic polysaccharide (B) contained in the core.

The pharmaceutical dosage form according to the invention comprises a polyalkylene oxide (C) having a weight average molecular weight of at least 200,000 g/mol.

In a preferred embodiment, the polyalkylene oxide (C) has a weight average molecular weight (M_{W}) or viscosity average molecular weight (M_{η}) of at least 500,000 g/mol, preferably at least 1,000,000 g/mol or at least 2,500,000 g/mol, more preferably in the range of about 1,000,000 g/mol to about 15,000,000 g/mol, and most preferably in the range of about 5,000,000 g/mol to about 10,000,000 g/mol. Suitable methods to determine M_{W} and M_{η} are known to a person skilled in the art. M_{η} is preferably determined by rheological measurements, whereas M_{W} can be determined by gel permeation chromatography (GPC).

Preferably, the molecular weight dispersity M_{W}/M_{η} of polyalkylene oxide (C) is within the range of 2.5±2.0, more preferably 2.5±1.5, still more preferably 2.5±1.0, yet more preferably 2.5±0.8, most preferably 2.5±0.6, and in particular 2.5±0.4.

The polyalkylene oxide (C) preferably has a viscosity at 25° C. of 30 to 17,600 cP, more preferably 55 to 17,600 cP, still more preferably 600 to 17,600 cP and most preferably 4,500 to 17,600 cP, measured in a 5 wt.-% aqueous solution using a model RVF Brookfield viscosimeter (spindle no. 2/rotational speed 2 rpm); of 400 to 4,000 cP, more preferably 400 to 800 cP or 2,000 to 4,000 cP, measured on a 2 wt.-% aqueous solution using the stated viscosimeter (spindle no. 1 or 3/rotational speed 10 rpm); or of 1,650 to 10,000 cP, more preferably 1,650 to 5,500 cP, 5,500 to 7,500 cP or 7,500 to 10,000 cP, measured on a 1 wt.-% aqueous solution using the stated viscosimeter (spindle no. 2/rotational speed 2 rpm).

Preferably, the polyalkylene oxide (C) is selected from polymethylene oxide, polyethylene oxide and polypropylene oxide, or copolymers thereof. Preferably, the polyalkylene oxide (C) is a polyethylene oxide.

The weight content of the polyalkylene oxide (C) is within the range of from 20 to 80 wt.-%, based on the total weight of the pharmaceutical dosage form. Preferably, the weight content of the polyalkylene oxide (C) is within the range of 50±20 wt.-%, based on the total weight of the dosage form. Preferably, the weight content of the polyalkylene oxide (C) is within the range of 50±30 wt.-%, more preferably 50±27 wt.-%, still more preferably 50±24 wt.-%, yet more preferably 50±21 wt.-%, even more preferably 50±18 wt.-%, and most preferably 50±15 wt.-%, based on the total weight of the dosage form.

Polyalkylene oxide (C) may comprise a single polyalkylene oxide having a particular average molecular weight, or a mixture (blend) of different polymers, such as two, three, four or five polymers, e.g., polymers of the same chemical nature but different average molecular weight, polymers of different chemical nature but same average molecular weight, or polymers of different chemical nature as well as different molecular weight.

For the purpose of the specification, a polyalkylene glycol has a molecular weight of up to 20,000 g/mol whereas a polyalkylene oxide has a molecular weight of more than 20,000 g/mol. In a preferred embodiment, the weight average over all molecular weights of all polyalkylene oxides that are contained in the pharmaceutical dosage form is at least 200,000 g/mol. Thus, polyalkylene glycols, if any, are preferably not taken into consideration when determining the weight average molecular weight of polyalkylene oxide (C).

In a preferred embodiment, polyalkylene oxide (C) is homogeneously distributed in the pharmaceutical dosage form according to the invention. Preferably, the opioid (A) and polyalkylene oxide (C) are intimately homogeneously distributed in the pharmaceutical dosage form so that the pharmaceutical dosage form does not contain any segments where either opioid (A) is present in the absence of polyalkylene oxide (C) or where polyalkylene oxide (C) is present in the absence of opioid (A).

When the pharmaceutical dosage form is film coated, the polyalkylene oxide (C) is preferably homogeneously distributed in the core of the pharmaceutical dosage form, i.e. the film coating preferably does not contain polyalkylene oxide (C). Nonetheless, the film coating as such may of course contain one or more polymers, which however, preferably differ from the polyalkylene oxide (C) contained in the core.

The polyalkylene oxide (C) may be combined with one or more different polymers selected from the group consisting of polyethylene, polypropylene, polyvinyl chloride, polycarbonate, polystyrene, polyvinylpyrrolidone, poly(alk)acrylate, poly(hydroxy fatty acids), such as for example poly(3-hydroxybutyrate-co-3-hydroxyvalerate) (Biopol), poly(hydroxyvaleric acid); polycaprolactone, polyvinyl alcohol, polyesteramide, polyethylene succinate, polylactone, polyglycolide, polyurethane, polyamide, polylactide, polyacetal (for example polysaccharides optionally with modified side chains), polylactide/glycolide, polylactone, polyglycolide, polyorthoester, polyanhydride, block polymers of polyethylene glycol and polybutylene terephthalate (Polyactive®), polyanhydride (Polifeprosan), copolymers thereof, block-copolymers thereof, and mixtures of at least two of the stated polymers, or other polymers with the above characteristics.

In a preferred embodiment, the dosage form according to the invention additionally comprises a non-ionic polysaccharide selected from the group consisting of methylcellulose, ethylcellulose, propylcellulose, hydroxyethylcellulose, hydroxypropylcellulose and hydroxypropylmethylcellulose.

Preferably, the weight content of the non-ionic polysaccharide is within the range of 10±9 wt.-%, more preferably 10±8 wt.-%, still more preferably 10±7 wt.-%, yet more preferably 10±6 wt.-%, even more preferably 10±5 wt.-%, and most preferably 10±4 wt.-%, based on the total weight of the dosage form.

In another preferred embodiment, the pharmaceutical dosage form according to the invention does not contain any further polymer besides the anionic polysaccharide (B), the polyalkylene oxide (C) and optionally, a polyethylene glycol (as plasticizer).

In a preferred embodiment, the relative weight ratio of the polyalkylene oxide (C) to the nonionic polysaccharide is within the range of from 4:1 to 1:4, more preferably 3.5:1 to 1:3.5, still more preferably 3:1 to 1:3, yet more preferably 2.5:1 to 1:2.5, most preferably 2:1 to 1:2 and in particular 1.5:1 to 1:1.5.

In a preferred embodiment, the relative weight ratio of the polyalkylene oxide (C) to the anionic polysaccharide (B) is within the range of from 8:1 to 1:1, more preferably 7:1 to 1:1, still more preferably 6:1 to 1.5:1, yet more preferably 5:1 to 1.5:1, most preferably 4:1 to 2:1 and in particular 3:1 to 2:1.

In a preferred embodiment, the relative weight ratio of the polyalkylene oxide (C) to the opioid (A) is within the range of from 8:1 to 1:1, more preferably 7:1 to 1:1, still more preferably 6:1 to 1.5:1, yet more preferably 5:1 to 1.5:1, most preferably 4:1 to 2:1 and in particular 3:1 to 2:1.

In a preferred embodiment, the relative weight ratio of the opioid (A) to the anionic polysaccharide (B) is within the range of from 4:1 to 1:4, more preferably 3.5:1 to 1:3.5, still more preferably 3:1 to 1:3, yet more preferably 2.5:1 to 1:2.5, most preferably 2:1 to 1:2 and in particular 1.5:1 to 1:1.5.

Besides the opioid (A), the anionic polysaccharide (B) and the polyalkylene oxide (C) the pharmaceutical dosage form according to the invention may contain further ingredients, e.g. one or more conventional pharmaceutical excipient(s), e.g. fillers, glidants, binding agents, granulating agents, anti-caking agents, lubricants, flavors, dyes, and/or preservatives.

In a preferred embodiment, the dosage form according to the invention additionally comprises a plasticizer preferably selected from the group consisting of polyalkylene glycol, triacetin, fatty acids, fatty acid esters, waxes and microcrystalline waxes. Particularly preferred plasticizers are polyethylene glycols, such as PEG 6000.

Preferably, the weight content of the plasticizer is preferably within the range of 10±7.5 wt.-%, based on the total weight of the dosage form. Preferably, the weight content of the plasticizer is within the range of 12±11 wt.-%, more preferably 12±10 wt.-%, still more preferably 12±9 wt.-%, yet more preferably 12±8 wt.-%, even more preferably 12±7 wt.-%, and most preferably 12±6 wt.-%, based on the total weight of the dosage form

In a preferred embodiment, the dosage form according to the invention, which additionally comprises an antioxidant preferably selected from the group consisting of ascorbic acid, salts of ascorbic acid, butylhydroxyanisole (BHA), butylhydroxytoluene (BHT), monothioglycerol, phosphorous acid, α-tocopherol, α-tocopheryl acetate, coniferyl benzoate, nordihydroguajaretic acid, gallus acid esters, and sodium bisulfate. A particularly preferred antioxidant is α-tocopherol.

Preferably, the weight content of the antioxidant is within the range of 1.00±0.95 wt.-%, based on the total weight of the dosage form. Preferably, the weight content of the antioxidant is within the range of 0.25±0.24 wt.-%, more preferably 0.25±0.21 wt.-%, still more preferably 0.25±0.18 wt.-%, yet more preferably 0.25±0.15 wt.-%, even more preferably 0.25±0.12 wt.-%, and most preferably 0.25±0.09 wt.-%, based on the total weight of the dosage form.

In a preferred embodiment, the dosage form according to the invention additionally comprises an acid, preferably selected from the group consisting of citric acid, fumaric acid, malic acid, maleic acid and tartaric acid. Citric acid is particularly preferred.

Preferably, the acid is essentially present in its free, i.e. acidic form. Thus, preferably, the acid is not part of a salt that is formed between the opioid (A) and the acid. In case that the opioid (A) is present in form of an acid addition salt, e.g. as hydrochloride or bitartrate, the acid is to be regarded as a distinct component which is separate from the hydrochloric acid and the tartaric acid, respectively.

Preferably, the weight content of the acid is within the range of 1.00±0.95 wt.-%, based on the total weight of the dosage form. Preferably, the weight content of the acid is within the range of 0.80±0.75 wt.-%, more preferably 0.80±0.70 wt.-%, still more preferably 0.80±0.65 wt.-%, yet more preferably 0.80±0.60 wt.-%, even more preferably 0.80±0.55 wt.-%, and most preferably 0.80±0.50 wt.-%, based on the total weight of the dosage form.

The pharmaceutical dosage form according to the invention is preferably an oral dosage form, particularly a tablet. It is also possible, however, to administer the pharmaceutical dosage form via different routes and thus, the pharmaceutical dosage form may alternatively be adapted for buccal, lingual, rectal or vaginal administration. Implants are also possible. Preferably, the pharmaceutical dosage form is monolithic. Preferably, the pharmaceutical dosage form is neither in film form, nor multi-particulate.

In a preferred embodiment, the pharmaceutical dosage form according to the invention is a round tablet. Tablets of this embodiment preferably have a diameter in the range of about 1 mm to about 30 mm, in particular in the range of about 2 mm to about 25 mm, more in particular about 5 mm to about 23 mm, even more in particular about 7 mm to about 13 mm; and a thickness in the range of about 1.0 mm to about 12 mm, in particular in the range of about 2.0 mm to about 10 mm, even more in particular from 3.0 mm to about 9.0 mm, even further in particular from about 4.0 mm to about 8.0 mm.

In another preferred embodiment, the pharmaceutical dosage form according to the invention is an oblong tablet. Tablets of this embodiment preferably have a lengthwise extension (longitudinal extension) of about 1 mm to about 30 mm, in particular in the range of about 2 mm to about 25 mm, more in particular about 5 mm to about 23 mm, even more in particular about 7 mm to about 20 mm; and a thickness in the range of about 1.0 mm to about 12 mm, in particular in the range of about 2.0 mm to about 10 mm, even more in particular from 3.0 mm to about 9.0 mm, even further in particular from about 4.0 mm to about 8.0 mm.

The pharmaceutical dosage form according to the invention has preferably a weight in the range of 0.01 to 1.5 g, more preferably in the range of 0.05 to 1.2 g, still more preferably in the range of 0.1 g to 1.0 g, yet more preferably in the range of 0.2 g to 0.9 g, and most preferably in the range of 0.25 g to 0.8 g.

The pharmaceutical dosage form of the invention can optionally be provided, partially or completely, with a conventional coating. The dosage forms of the present invention are preferably film coated with conventional film coating compositions.

Suitable coating materials are commercially available, e.g. under the trademarks Opadry® and Eudragit®.

Examples of suitable materials include cellulose esters and cellulose ethers, such as methylcellulose (MC), hydroxypropylmethylcellulose (HPMC), hydroxypropylcellulose (HPC), hydroxyethylcellulose (HEC), sodium carboxymethylcellulose (Na-CMC), ethylcellulose (EC), cellulose acetate phthalate (CAP), hydroxypropylmethylcellulose phthalate (HPMCP); poly(meth)acrylates, such as aminoalkylmethacrylate copolymers, ethylacrylate methylmethacrylate copolymers, methacrylic acid methylmethacrylate copolymers, methacrylic acid methylmethacrylate copolymers; vinyl polymers, such as polyvinylpyrrolidone, polyvinylacetatephthalate, polyvinyl alcohol, polyvinylacetate; and natural film formers, such as shellack.

In a particularly preferred embodiment, the coating is water-soluble. In a preferred embodiment, the coating is based on polyvinyl alcohol, such as polyvinyl alcohol-part. Hydrolyzed, and may additionally contain polyethylene glycol, such as macrogol 3350, and/or pigments. In another preferred embodiment, the coating is based on hydroxypropylmethylcellulose, preferably hypromellose type 2910 having a viscosity of 3 to 15 mPas.

The coating can be resistant to gastric juices and dissolve as a function of the pH value of the release environment. By means of this coating, it is possible to ensure that the pharmaceutical dosage form according to the invention passes through the stomach undissolved and the active ingredient is only released in the intestines. The coating which is resistant to gastric juices preferably dissolves at a pH value of between 5 and 7.5. Corresponding materials and methods for the delayed release of active ingredients and for the application of coatings which are resistant to gastric juices are known to the person skilled in the art, for example from “Coated Pharmaceutical dosage forms—Fundamentals, Manufacturing Techniques, Biopharmaceutical Aspects, Test Methods and Raw Materials” by Kurt H. Bauer, K. Lehmann, Hermann P. Osterwald, Rothgang, Gerhart, 1st edition, 1998, Medpharm Scientific Publishers.

The coating can also be applied e.g. to improve the aesthetic impression and/or the taste of the dosage forms and the ease with which they can be swallowed. Coating the dosage forms of the present invention can also serve other purposes, e.g. improving stability and shelf-life. Suitable coating formulations comprise a film forming polymer such as, for example, polyvinyl alcohol or hydroxypropyl methylcellulose, e.g. hypromellose, a plasticizer such as, for example, a glycol, e.g. propylene glycol or polyethylene glycol, an opacifier, such as, for example, titanium dioxide, and a film smoothener, such as, for example, talc. Suitable coating solvents are water as well as organic solvents. Examples of organic solvents are alcohols, e.g. ethanol or isopropanol, ketones, e.g. acetone, or halogenated hydrocarbons, e.g. methylene chloride. Optionally, the coating can contain a therapeutically effective amount of one or more active ingredients to provide for an immediate release of said opioid (A) and thus for an immediate relief of the symptoms treated by said opioid (A). Coated dosage forms of the present invention are preferably prepared by first making the cores and subsequently coating said cores using conventional techniques, such as coating in a coating pan.

According to the invention, the opioid (A) is embedded in a controlled-release matrix comprising anionic polysaccharide (B) and polyalkylene oxide (C).

Controlled release of an active ingredient from an oral dosage form is known to a person skilled in the art. For the purpose of the specification, controlled release encompasses delayed release, retarded release, sustained release, prolonged release, and the like.

Controlled or prolonged release is understood according to the invention preferably to mean a release profile in which the opioid (A) is released over a relatively long period with reduced intake frequency with the purpose of extended therapeutic action. Preferably, the meaning of the term “prolonged release” is in accordance with the European guideline on the nomenclature of the release profile of pharmaceutical dosage forms (CHMP). This is achieved in particular with peroral administration. The expression “at least partially delayed or prolonged release” covers according to the invention any pharmaceutical dosage forms which ensure modified release of the opioids (A) contained therein. The pharmaceutical dosage forms preferably comprise coated or uncoated pharmaceutical dosage forms, which are produced with specific auxiliary substances, by particular processes or by a combination of the two possible options in order purposefully to change the release rate or location of release.

In the case of the pharmaceutical dosage forms according to the invention, the release time profile of a controlled release form may be modified e.g. as follows: extended release, repeat action release, prolonged release and sustained release.

For the purpose of the specification “controlled release” preferably means a product in which the release of active ingredient over time is controlled by the type and composition of the formulation. For the purpose of the specification “extended release” preferably means a product in which the release of active ingredient is delayed for a finite lag time, after which release is unhindered. For the purpose of the specification “repeat action release” preferably means a product in which a first portion of active ingredient is released initially, followed by at least one further portion of active ingredient being released subsequently. For the purpose of the specification “prolonged release” preferably means a product in which the rate of release of active ingredient from the formulation after administration has been reduced over time, in order to maintain therapeutic activity, to reduce toxic effects, or for some other therapeutic purpose. For the purpose of the specification “sustained release” preferably means a way of formulating a medicine so that it is released into the body steadily, over a long period of time, thus reducing the dosing frequency. For further details, reference may be made, for example, to K. H. Bauer, Lehrbuch der Pharmazeutischen Technologie, 6th edition, WVG Stuttgart, 1999; and Eur. Ph.

Preferably the pharmaceutically dosage form provides a release of the opioid (A) after 1 hour of preferably at most 60%, more preferably at most 40%, yet more preferably at most 30%, still more preferably at most 20% and most preferably at most 17%. After 2 hour preferably at most 80%, more preferably at most 60%, yet more preferably at most 50%, still more preferably at most 40% and most preferably at most 32%. After 3 hour preferably at most 85%, more preferably at most 65%, yet more preferably at most 55%, still more preferably at most 48% and most preferably at most 42%. After 4 hour preferably at most 90%, more preferably at most 75%, yet more preferably at most 65%, still more preferably at most 55% and most preferably at most 49%. After 7 hour preferably at most 95%, more preferably at most 85%, yet more preferably at most 80%, still more preferably at most 70% and most preferably at most 68%. After 10 hour preferably at most 99%, more preferably at most 90%, yet more preferably at most 88%, still more preferably at most 83% and most preferably at most 80%. After 13 hour preferably at most 99%, more preferably at most 95%, yet more preferably at most 93%, still more preferably at most 91% and most preferably at most 89%.

In a preferred embodiment, the dosage form according to the invention which has released under in vitro conditions:

after 1 h at most 40 wt.-%,
after 2 h at most 55 wt.-%,
after 3 h at most 70 wt.-%, and
after 4 h at most 85 wt.-%
of the total content of the opioid (A) that was originally contained in the dosage form.

Suitable in vitro conditions are known to the skilled artisan. In this regard it can be referred to, e.g., the Eur. Ph. Preferably, the release profile is measured under the following conditions: Paddle apparatus equipped with sinker, 75 rpm, 37±5° C., 900 mL simulated intestinal fluid pH 6.8 (phosphate buffer) or pH 4.5. In a preferred embodiment, the rotational speed of the paddle is increased to 100 rpm.

Preferably, the dosage form according to the invention is for use in therapy, wherein the dosage form is administered once daily or twice daily. Thus, in a preferred embodiment, the pharmaceutical dosage form according to the invention is adapted for administration once daily. In another preferred embodiment, the pharmaceutical dosage form according to the invention is adapted for administration twice daily. In still another preferred embodiment, the pharmaceutical dosage form according to the invention is adapted for administration thrice daily.

For the purpose of the specification, “twice daily” means equal or nearly equal time intervals, i.e., about every 12 hours, or different time intervals, e.g., 8 and 16 hours or 10 and 14 hours, between the individual administrations.

For the purpose of the specification, “thrice daily” means equal or nearly equal time intervals, i.e., about every 8 hours, or different time intervals, e.g., 6, 6 and 12 hours; or 7, 7 and 10 hours, between the individual administrations.

In a preferred embodiment, the pharmaceutical dosage form according to the invention contains no substances which irritate the nasal passages and/or pharynx, i.e. substances which, when administered via the nasal passages and/or pharynx, bring about a physical reaction which is either so unpleasant for the patient that he/she does not wish to or cannot continue administration, for example burning, or physiologically counteracts taking of the corresponding active ingredient, for example due to increased nasal secretion or sneezing. Further examples of substances which irritate the nasal passages and/or pharynx are those which cause burning, itching, urge to sneeze, increased formation of secretions or a combination of at least two of these stimuli. Corresponding substances and the quantities thereof which are conventionally to be used are known to the person skilled in the art. Some of the substances which irritate the nasal passages and/or pharynx are accordingly based on one or more constituents or one or more plant parts of a hot substance drug. Corresponding hot substance drugs are known per se to the person skilled in the art and are described, for example, in “Pharmazeutische Biologie—Drogen und ihre Inhaltsstoffe” by Prof. Dr. Hildebert Wagner, 2nd., revised edition, Gustav Fischer Verlag, Stuttgart-New York, 1982, pages 82 et seq. The corresponding description is hereby introduced as a reference and is deemed to be part of the disclosure.

The pharmaceutical dosage form according to the invention furthermore preferably contains no antagonists for the opioid (A), preferably no antagonists against psychotropic substances, in particular no antagonists against opioids (A). Antagonists suitable for a given opioid (A) are known to the person skilled in the art and may be present as such or in the form of corresponding derivatives, in particular esters or ethers, or in each case in the form of corresponding physiologically acceptable compounds, in particular in the form of the salts or solvates thereof. The pharmaceutical dosage form according to the invention preferably contains no antagonists selected from among the group comprising naloxone, naltrexone, nalmefene, nalide, nalmexone, nalorphine or naluphine, in each case optionally in the form of a corresponding physiologically acceptable compound, in particular in the form of a base, a salt or solvate; and no neuroleptics, for example a compound selected from among the group comprising haloperidol, promethacine, fluphenazine, perphenazine, levomepromazine, thioridazine, perazine, chlorpromazine, chlorprothixine, zuclopenthixol, flupentixol, prothipendyl, zotepine, benperidol, pipamperone, melperone and bromperidol.

The pharmaceutical dosage form according to the invention furthermore preferably contains no emetic. Emetics are known to the person skilled in the art and may be present as such or in the form of corresponding derivatives, in particular esters or ethers, or in each case in the form of corresponding physiologically acceptable compounds, in particular in the form of the salts or solvates thereof. The pharmaceutical dosage form according to the invention preferably contains no emetic based on one or more constituents of ipecacuanha (ipecac) root, for example based on the constituent emetine, as are, for example, described in “Pharmazeutische Biologie—Drogen und ihre Inhaltsstoffe” by Prof. Dr. Hildebert Wagner, 2nd, revised edition, Gustav Fischer Verlag, Stuttgart, New York, 1982. The corresponding literature description is hereby introduced as a reference and is deemed to be part of the disclosure. The pharmaceutical dosage form according to the invention preferably also contains no apomorphine as an emetic.

Finally, the pharmaceutical dosage form according to the invention preferably also contains no bitter substance. Bitter substances and the quantities effective for use may be found in US-2003/0064099 A1, the corresponding disclosure of which should be deemed to be the disclosure of the present application and is hereby introduced as a reference. Examples of bitter substances are aromatic oils, such as peppermint oil, eucalyptus oil, bitter almond oil, menthol, fruit aroma substances, aroma substances from lemons, oranges, limes, grapefruit or mixtures thereof, and/or denatonium benzoate.

The pharmaceutical dosage form according to the invention accordingly preferably contains neither substances which irritate the nasal passages and/or pharynx, nor antagonists for the opioid (A), nor emetics, nor bitter substances.

The pharmaceutical dosage form according to the invention has a breaking strength of at least 300 N, preferably at least 500 N.

The pharmaceutical dosage form according to the invention is preferably tamper-resistant. Preferably, tamper-resistance is achieved based on the mechanical properties of the pharmaceutical dosage form so that comminution is avoided or at least substantially impeded. According to the invention, the term comminution means the pulverization of the pharmaceutical dosage form using conventional means usually available to an abuser, for example a pestle and mortar, a hammer, a mallet or other conventional means for pulverizing under the action of force. Thus, tamper-resistance preferably means that pulverization of the pharmaceutical dosage form using conventional means is avoided or at least substantially impeded.

Preferably, the mechanical properties of the pharmaceutical dosage form according to the invention, particularly its breaking strength, substantially rely on the presence and spatial distribution of anionic polysaccharide (B) and polyalkylene oxide (C), although their mere presence does typically not suffice in order to achieve said properties. The advantageous mechanical properties of the pharmaceutical dosage form according to the invention may not automatically be achieved by simply processing opioid (A), anionic polysaccharide (B), polyalkylene oxide (C), and optionally further excipients by means of conventional methods for the preparation of pharmaceutical dosage forms. In fact, usually suitable apparatuses must be selected for the preparation and critical processing parameters must be adjusted, particularly pressure/force, temperature and time. Thus, even if conventional apparatuses are used, the process protocols usually must be adapted in order to meet the required criteria.

In general, the dosage forms exhibiting the desired properties may be obtained only if, during preparation of the dosage form,
suitable components
in suitable amounts

are exposed to
a sufficient pressure
at a sufficient temperature
for a sufficient period of time.

Thus, regardless of the apparatus used, the process protocols must be adapted in order to meet the required criteria. Therefore, the breaking strength is separable from the composition.

The pharmaceutical dosage form according to the invention has a breaking strength of at least 300 N, preferably at least 500 N, preferably at least 600 N, more preferably at least 700 N, still more preferably at least 800 N, yet more preferably at least 1000 N, most preferably at least 1250 N and in particular at least 1500 N.

The “breaking strength” (resistance to crushing) of a pharmaceutical dosage form is known to the skilled person. In this regard it can be referred to, e.g., W. A. Ritschel, Die Tablette, 2. Auflage, Editio Cantor Verlag Aulendorf, 2002; H Liebermann et al., Pharmaceutical dosage forms: Tablets, Vol. 2, Informa Healthcare; 2 edition, 1990; and Encyclopedia of Pharmaceutical Technology, Informa Healthcare; 1 edition.

For the purpose of the specification, the breaking strength is preferably defined as the amount of force that is necessary in order to fracture the pharmaceutical dosage form (=breaking force). Therefore, for the purpose of the specification the pharmaceutical dosage form does preferably not exhibit the desired breaking strength when it breaks, i.e., is fractured into at least two independent parts that are separated from one another. In another preferred embodiment, however, the pharmaceutical dosage form is regarded as being broken if the force decreases by 25% (threshold value) of the highest force measured during the measurement (see below).

The pharmaceutical dosage forms according to the invention are distinguished from conventional pharmaceutical dosage forms in that, due to their breaking strength, they cannot be pulverized by the application of force with conventional means, such as for example a pestle and mortar, a hammer, a mallet or other usual means for pulverization, in particular devices developed for this purpose (tablet crushers). In this regard “pulverization” means crumbling into small particles that would immediately release the opioid (A) in a suitable medium. Avoidance of pulverization virtually rules out oral or parenteral, in particular intravenous or nasal abuse.

Conventional tablets typically have a breaking strength well below 200 N in any direction of extension. The breaking strength of conventional round tablets may be estimated according to the following empirical formula: Breaking Strength [in N]=10× Diameter Of The Tablet [in mm]. Thus, according to said empirical formula, a round tablet having a breaking strength of at least 300 N would require a diameter of at least 30 mm). Such a tablet, however, could not be swallowed. The above empirical formula preferably does not apply to the pharmaceutical dosage forms of the invention, which are not conventional but rather special.

Further, the actual mean chewing force is about 220 N (cf., e.g., P. A. Proeschel et al., J Dent Res, 2002, 81(7), 464-468). This means that conventional tablets having a breaking strength well below 200 N may be crushed upon spontaneous chewing, whereas the pharmaceutical dosage forms according to the invention may not.

Still further, when applying a gravitational acceleration of about 9.81 m/s², 500 N correspond to a gravitational force of more than 50 kg, i.e. the pharmaceutical dosage forms according to the invention can preferably withstand a weight of more than 50 kg without being pulverized.

Methods for measuring the breaking strength of a pharmaceutical dosage form are known to the skilled artisan. Suitable devices are commercially available.

For example, the breaking strength (resistance to crushing) can be measured in accordance with the Eur. Ph. 5.0, 2.9.8 or 6.0, 2.09.08 “Resistance to Crushing of Tablets”. The test is intended to determine, under defined conditions, the resistance to crushing of tablets, measured by the force needed to disrupt them by crushing. The apparatus consists of 2 jaws facing each other, one of which moves towards the other. The flat surfaces of the jaws are perpendicular to the direction of movement. The crushing surfaces of the jaws are flat and larger than the zone of contact with the tablet. The apparatus is calibrated using a system with a precision of 1 Newton. The tablet is placed between the jaws, taking into account, where applicable, the shape, the break-mark and the inscription; for each measurement the tablet is oriented in the same way with respect to the direction of application of the force (and the direction of extension in which the breaking strength is to be measured). The measurement is carried out on 10 tablets, taking care that all fragments of tablets have been removed before each determination. The result is expressed as the mean, minimum and maximum values of the forces measured, all expressed in Newton.

A similar description of the breaking strength (breaking force) can be found in the USP. The breaking strength can alternatively be measured in accordance with the method described therein where it is stated that the breaking strength is the force required to cause a tablet to fail (i.e., break) in a specific plane. The tablets are generally placed between two platens, one of which moves to apply sufficient force to the tablet to cause fracture. For conventional, round (circular cross-section) tablets, loading occurs across their diameter (sometimes referred to as diametral loading), and fracture occurs in the plane. The breaking force of tablets is commonly called hardness in the pharmaceutical literature; however, the use of this term is misleading. In material science, the term hardness refers to the resistance of a surface to penetration or indentation by a small probe. The term crushing strength is also frequently used to describe the resistance of tablets to the application of a compressive load. Although this term describes the true nature of the test more accurately than does hardness, it implies that tablets are actually crushed during the test, which is often not the case.

Alternatively, the breaking strength (resistance to crushing) can be measured in accordance with WO 2005/016313, WO 2005/016314, and WO 2006/082099, which can be regarded as a modification of the method described in the Eur. Ph. The apparatus used for the measurement is preferably a “Zwick Z 2.5” materials tester, Fₘₐₓ=2.5 kN with a maximum draw of 1150 mm, which should be set up with one column and one spindle, a clearance behind of 100 mm and a test speed adjustable between 0.1 and 800 mm/min together with testControl software. Measurement is performed using a pressure piston with screw-in inserts and a cylinder (diameter 10 mm), a force transducer, Fₘₐₓ. 1 kN, diameter=8 mm, class 0.5 from 10 N, class 1 from 2 N to ISO 7500-1, with manufacturer's test certificate M according to DIN 55350-18 (Zwick gross force Fₘₐₓ=1.45 kN) (all apparatus from Zwick GmbH & Co. KG, Ulm, Germany) with Order No BTC-FR 2.5 TH. D09 for the tester, Order No BTC-LC 0050N. P01 for the force transducer, Order No BO 70000 S06 for the centering device.

In a preferred embodiment of the invention, the breaking strength is measured by means of a breaking strength tester e.g. Sotax®, type HT100 or type HT1 (Allschwil, Switzerland). Both, the Sotax® HT100 and the Sotax® HT1 can measure the breaking strength according to two different measurement principles: constant speed (where the test jaw is moved at a constant speed adjustable from 5-200 mm/min) or constant force (where the test jaw increases force linearly adjustable from 5-100 N/sec). In principle, both measurement principles are suitable for measuring the breaking strength of the pharmaceutical dosage form according to the invention. Preferably, the breaking strength is measured at constant speed, preferably at a constant speed of 120 mm/min.

In a preferred embodiment, the pharmaceutical dosage form is regarded as being broken if it is fractured into at least two separate pieces.

The pharmaceutical dosage form according to the invention preferably exhibits mechanical strength over a wide temperature range, in addition to the breaking strength (resistance to crushing) optionally also sufficient hardness, impact resistance, impact elasticity, tensile strength and/or modulus of elasticity, optionally also at low temperatures (e.g. below −24° C., below −40° C. or in liquid nitrogen), for it to be virtually impossible to pulverize by spontaneous chewing, grinding in a mortar, pounding, etc. Thus, preferably, in direction of extension E₁ the comparatively high breaking strength of the pharmaceutical dosage form according to the invention is maintained even at low or very low temperatures, e.g., when the pharmaceutical dosage form is initially chilled to increase its brittleness, for example to temperatures below −25° C., below −40° C. or even in liquid nitrogen.

The pharmaceutical dosage form according to the invention is characterized by a certain degree of breaking strength. This does not mean that the pharmaceutical dosage form must also exhibit a certain degree of hardness. Hardness and breaking strength are different physical properties. Therefore, the tamper resistance of the pharmaceutical dosage form does not necessarily depend on the hardness of the pharmaceutical dosage form. For instance, due to its breaking strength, impact strength, elasticity modulus and tensile strength, respectively, the pharmaceutical dosage form can preferably be deformed, e.g. plastically, when exerting an external force, for example using a hammer, but cannot be pulverized, i.e., crumbled into a high number of fragments. In other words, the pharmaceutical dosage form according to the invention is characterized by a certain degree of breaking strength, but not necessarily also by a certain degree of form stability.

Therefore, in the meaning of the specification, a pharmaceutical dosage form that is deformed when being exposed to a force in a particular direction of extension but that does not break (plastic deformation or plastic flow) is preferably to be regarded as having the desired breaking strength in said direction of extension.

Preferably, the dosage form according to the invention provides resistance against extraction of the opioid (A) in water at room temperature such that when treating the dosage form for 30 min with 30 mL of water at room temperature, the extracted amount of opioid (A) is not more than 25 wt.-%, more preferably not more than 22.5 wt.-%, still more preferably not more than 20 wt.-%, yet more preferably not more than 17.5 wt.-%, even more preferably not more than 15 wt.-%, and most preferably not more than 12.5 wt.-% of the total content of the opioid (A) that was originally contained in the dosage form.

Preferably, the dosage form according to the invention provides resistance against extraction of the opioid (A) in water at 100° C. such that when treating the dosage form for 30 min with 30 mL of water at 100° C., the extracted amount of opioid (A) is not more than 40 wt.-%, more preferably not more than 37.5 wt.-%, still more preferably not more than 35 wt.-%, yet more preferably not more than 32.5 wt.-%, even more preferably not more than 30 wt.-%, and most preferably not more than 27.5 wt.-%, of the total content of the opioid (A) that was originally contained in the dosage form.

Preferably, the dosage form according to the invention provides resistance against extraction of the opioid (A) in ethanol at room temperature such that when treating the dosage form for 30 min with 30 mL of ethanol at room temperature, the extracted amount of opioid (A) is not more than 20 wt.-%, more preferably not more than 17.5 wt.-%, still more preferably not more than 15 wt.-%, yet more preferably not more than 12.5 wt.-%, even more preferably not more than 10 wt.-%, and most preferably not more than 7.5 wt.-%, of the total content of the opioid (A) that was originally contained in the dosage form.

Particularly preferred compositions of the dosage form according to the invention are compiled as embodiments A¹ to A⁴⁸ in the tables here below (according to these embodiment, the dosage form according to the invention comprises the specified ingredients in the specified quantities but may additionally comprise further ingredients):

| Ingredient [wt.-%] | A¹ | A² | A³ | A⁴ | A⁵ | A⁶ |
|---|---|---|---|---|---|---|
| Opioid (A) | 19 ± 15 | 19 ± 13 | 19 ± 11 | 19 ± 9 | 19 ± 7 | 19 ± 5 |
| Anionic | 20 ± 15 | 20 ± 13 | 20 ± 11 | 20 ± 9 | 20 ± 7 | 20 ± 5 |
| polysaccharide (B) | | | | | | |
| Polyalkylene oxide | 50 ± 30 | 50 ± 27 | 50 ± 24 | 50 ± 21 | 50 ± 18 | 50 ± 15 |
| (C) | | | | | | |
| Ingredient [wt.-%] | A⁷ | A⁸ | A⁹ | A¹⁰ | A¹¹ | A¹² |
| Opioid (A) | 19 ± 15 | 19 ± 13 | 19 ± 11 | 19 ± 9 | 19 ± 7 | 19 ± 5 |
| Croscarmellose or | 20 ± 15 | 20 ± 13 | 20 ± 11 | 20 ± 9 | 20 ± 7 | 20 ± 5 |
| salt thereof | | | | | | |
| PEO M_{w} ≧ 500,000 | 50 ± 30 | 50 ± 27 | 50 ± 24 | 50 ± 21 | 50 ± 18 | 50 ± 15 |
| g/mol | | | | | | |
| Ingredient [wt.-%] | A¹³ | A¹⁴ | A¹⁵ | A¹⁶ | A¹⁷ | A¹⁸ |
| Opioid (A) | 19 ± 15 | 19 ± 13 | 19 ± 11 | 19 ± 9 | 19 ± 7 | 19 ± 5 |
| Anionic | 20 ± 15 | 20 ± 13 | 20 ± 11 | 20 ± 9 | 20 ± 7 | 20 ± 5 |
| polysaccharide (B) | | | | | | |
| Polyalkylene oxide | 50 ± 30 | 50 ± 27 | 50 ± 24 | 50 ± 21 | 50 ± 18 | 50 ± 15 |
| (C) | | | | | | |
| Plasticizer | 12 ± 11 | 12 ± 10 | 12 ± 9 | 12 ± 8 | 12 ± 7 | 12 ± 6 |
| Ingredient [wt.-%] | A¹⁹ | A²⁰ | A²¹ | A²² | A²³ | A²⁴ |
| Opioid (A) | 19 ± 15 | 19 ± 13 | 19 ± 11 | 19 ± 9 | 19 ± 7 | 19 ± 5 |
| Croscarmellose or | 20 ± 15 | 20 ± 13 | 20 ± 11 | 20 ± 9 | 20 ± 7 | 20 ± 5 |
| salt thereof | | | | | | |
| PEO M_{w} ≧ 500,000 | 50 ± 30 | 50 ± 27 | 50 ± 24 | 50 ± 21 | 50 ± 18 | 50 ± 15 |
| g/mol | | | | | | |
| Polyethylene glycol | 12 ± 11 | 12 ± 10 | 12 ± 9 | 12 ± 8 | 12 ± 7 | 12 ± 6 |
| Ingredient [wt.-%] | A²⁵ | A²⁶ | A²⁷ | A²⁸ | A²⁹ | A³⁰ |
| Opioid (A) | 19 ± 15 | 19 ± 13 | 19 ± 11 | 19 ± 9 | 19 ± 7 | 19 ± 5 |
| Anionic | 20 ± 15 | 20 ± 13 | 20 ± 11 | 20 ± 9 | 20 ± 7 | 20 ± 5 |
| polysaccharide (B) | | | | | | |
| Polyalkylene oxide | 50 ± 30 | 50 ± 27 | 50 ± 24 | 50 ± 21 | 50 ± 18 | 50 ± 15 |
| (C) | | | | | | |
| Plasticizer | 12 ± 11 | 12 ± 10 | 12 ± 9 | 12 ± 8 | 12 ± 7 | 12 ± 6 |
| Antioxidant | 0.25 ± 0.24 | 0.25 ± 0.21 | 0.25 ± 0.18 | 0.25 ± 0.15 | 0.25 ± 0.12 | 0.25 ± 0.09 |
| Ingredient [wt.-%] | A³¹ | A³² | A³³ | A³⁴ | A³⁵ | A³⁶ |
| Opioid (A) | 19 ± 15 | 19 ± 13 | 19 ± 11 | 19 ± 9 | 19 ± 7 | 19 ± 5 |
| Croscarmellose or | 20 ± 15 | 20 ± 13 | 20 ± 11 | 20 ± 9 | 20 ± 7 | 20 ± 5 |
| salt thereof | | | | | | |
| PEO M_{w} ≧ 500,000 | 50 ± 30 | 50 ± 27 | 50 ± 24 | 50 ± 21 | 50 ± 18 | 50 ± 15 |
| g/mol | | | | | | |
| Polyethylene glycol | 12 ± 11 | 12 ± 10 | 12 ± 9 | 12 ± 8 | 12 ± 7 | 12 ± 6 |
| α-tocopherol | 0.25 ± 0.24 | 0.25 ± 0.21 | 0.25 ± 0.18 | 0.25 ± 0.15 | 0.25 ± 0.12 | 0.25 ± 0.09 |
| Ingredient [wt.-%] | A³⁷ | A³⁸ | A³⁹ | A⁴⁰ | A⁴¹ | A⁴² |
| Opioid (A) | 19 ± 15 | 19 ± 13 | 19 ± 11 | 19 ± 9 | 19 ± 7 | 19 ± 5 |
| Anionic | 20 ± 15 | 20 ± 13 | 20 ± 11 | 20 ± 9 | 20 ± 7 | 20 ± 5 |
| polysaccharide (B) | | | | | | |
| Polyalkylene oxide | 50 ± 30 | 50 ± 27 | 50 ± 24 | 50 ± 21 | 50 ± 18 | 50 ± 15 |
| (C) | | | | | | |
| Plasticizer | 12 ± 11 | 12 ± 10 | 12 ± 9 | 12 ± 8 | 12 ± 7 | 12 ± 6 |
| Antioxidant | 0.25 ± 0.24 | 0.25 ± 0.21 | 0.25 ± 0.18 | 0.25 ± 0.15 | 0.25 ± 0.12 | 0.25 ± 0.09 |
| Acid | 0.80 ± 0.75 | 0.80 ± 0.70 | 0.80 ± 0.65 | 0.80 ± 0.60 | 0.80 ± 0.55 | 0.80 ± 0.50 |
| Ingredient [wt.-%] | A⁴³ | A⁴⁴ | A⁴⁵ | A⁴⁶ | A⁴⁷ | A⁴⁸ |
| Opioid (A) | 19 ± 15 | 19 ± 13 | 19 ± 11 | 19 ± 9 | 19 ± 7 | 19 ± 5 |
| Croscarmellose or | 20 ± 15 | 20 ± 13 | 20 ± 11 | 20 ± 9 | 20 ± 7 | 20 ± 5 |
| salt thereof | | | | | | |
| PEO M_{w} ≧ 500,000 | 50 ± 30 | 50 ± 27 | 50 ± 24 | 50 ± 21 | 50 ± 18 | 50 ± 15 |
| g/mol | | | | | | |
| Polyethylene glycol | 12 ± 11 | 12 ± 10 | 12 ± 9 | 12 ± 8 | 12 ± 7 | 12 ± 6 |
| α-tocopherol | 0.25 ± 0.24 | 0.25 ± 0.21 | 0.25 ± 0.18 | 0.25 ± 0.15 | 0.25 ± 0.12 | 0.25 ± 0.09 |
| Citric acid | 0.80 ± 0.75 | 0.80 ± 0.70 | 0.80 ± 0.65 | 0.80 ± 0.60 | 0.80 ± 0.55 | 0.80 ± 0.50 |

PEO = Polyethylene oxide

Particularly preferred compositions of the dosage form according to the invention are compiled as embodiments B¹ to B⁴⁸ in the tables here below (according to these embodiment, the dosage form according to the invention comprises the specified ingredients in the specified quantities but may additionally comprise further ingredients):

| Ingredient [wt.-%] | B¹ | B² | B³ | B⁴ | B⁵ | B⁶ |
|---|---|---|---|---|---|---|
| Opioid (A) | 19 ± 15 | 19 ± 13 | 19 ± 11 | 19 ± 9 | 19 ± 7 | 19 ± 5 |
| Anionic | 10 ± 9 | 10 ± 8 | 10 ± 7 | 10 ± 6 | 10 ± 5 | 10 ± 4 |
| polysaccharide (B) | | | | | | |
| Nonionic | 10 ± 9 | 10 ± 8 | 10 ± 7 | 10 ± 6 | 10 ± 5 | 10 ± 4 |
| polysaccharide | | | | | | |
| Polyalkylene oxide | 50 ± 30 | 50 ± 27 | 50 ± 24 | 50 ± 21 | 50 ± 18 | 50 ± 15 |
| (C) | | | | | | |
| Ingredient [wt.-%] | B⁷ | B⁸ | B⁹ | B¹⁰ | B¹¹ | B¹² |
| Opioid (A) | 19 ± 15 | 19 ± 13 | 19 ± 11 | 19 ± 9 | 19 ± 7 | 19 ± 5 |
| Croscarmellose or | 10 ± 9 | 10 ± 8 | 10 ± 7 | 10 ± 6 | 10 ± 5 | 10 ± 4 |
| salt thereof | | | | | | |
| HPMC | 10 ± 9 | 10 ± 8 | 10 ± 7 | 10 ± 6 | 10 ± 5 | 10 ± 4 |
| PEO M_{w} ≧ 500,000 | 50 ± 30 | 50 ± 27 | 50 ± 24 | 50 ± 21 | 50 ± 18 | 50 ± 15 |
| g/mol | | | | | | |
| Ingredient [wt.-%] | B¹³ | B¹⁴ | B¹⁵ | B¹⁶ | B¹⁷ | B¹⁸ |
| Opioid (A) | 19 ± 15 | 19 ± 13 | 19 ± 11 | 19 ± 9 | 19 ± 7 | 19 ± 5 |
| Anionic | 10 ± 9 | 10 ± 8 | 10 ± 7 | 10 ± 6 | 10 ± 5 | 10 ± 4 |
| polysaccharide (B) | | | | | | |
| Nonionic | 10 ± 9 | 10 ± 8 | 10 ± 7 | 10 ± 6 | 10 ± 5 | 10 ± 4 |
| polysaccharide | | | | | | |
| Polyalkylene oxide | 50 ± 30 | 50 ± 27 | 50 ± 24 | 50 ± 21 | 50 ± 18 | 50 ± 15 |
| (C) | | | | | | |
| Plasticizer | 12 ± 11 | 12 ± 10 | 12 ± 9 | 12 ± 8 | 12 ± 7 | 12 ± 6 |
| Ingredient [wt.-%] | B¹⁹ | B²⁰ | B²¹ | B²² | B²³ | B²⁴ |
| Opioid (A) | 19 ± 15 | 19 ± 13 | 19 ± 11 | 19 ± 9 | 19 ± 7 | 19 ± 5 |
| Croscarmellose or | 10 ± 9 | 10 ± 8 | 10 ± 7 | 10 ± 6 | 10 ± 5 | 10 ± 4 |
| salt thereof | | | | | | |
| HPMC | 10 ± 9 | 10 ± 8 | 10 ± 7 | 10 ± 6 | 10 ± 5 | 10 ± 4 |
| PEO M_{w} ≧ 500,000 | 50 ± 30 | 50 ± 27 | 50 ± 24 | 50 ± 21 | 50 ± 18 | 50 ± 15 |
| g/mol | | | | | | |
| Polyethylene glycol | 12 ± 11 | 12 ± 10 | 12 ± 9 | 12 ± 8 | 12 ± 7 | 12 ± 6 |
| Ingredient [wt.-%] | B²⁵ | B²⁶ | B²⁷ | B²⁸ | B²⁹ | B³⁰ |
| Opioid (A) | 19 ± 15 | 19 ± 13 | 19 ± 11 | 19 ± 9 | 19 ± 7 | 19 ± 5 |
| Anionic | 10 ± 9 | 10 ± 8 | 10 ± 7 | 10 ± 6 | 10 ± 5 | 10 ± 4 |
| polysaccharide (B) | | | | | | |
| Nonionic | 10 ± 9 | 10 ± 8 | 10 ± 7 | 10 ± 6 | 10 ± 5 | 10 ± 4 |
| polysaccharide | | | | | | |
| Polyalkylene oxide | 50 ± 30 | 50 ± 27 | 50 ± 24 | 50 ± 21 | 50 ± 18 | 50 ± 15 |
| (C) | | | | | | |
| Plasticizer | 12 ± 11 | 12 ± 10 | 12 ± 9 | 12 ± 8 | 12 ± 7 | 12 ± 6 |
| Antioxidant | 0.25 ± 0.24 | 0.25 ± 0.21 | 0.25 ± 0.18 | 0.25 ± 0.15 | 0.25 ± 0.12 | 0.25 ± 0.09 |
| Ingredient [wt.-%] | B³¹ | B³² | B³³ | B³⁴ | B³⁵ | B³⁶ |
| Opioid (A) | 19 ± 15 | 19 ± 13 | 19 ± 11 | 19 ± 9 | 19 ± 7 | 19 ± 5 |
| Croscarmellose or | 10 ± 9 | 10 ± 8 | 10 ± 7 | 10 ± 6 | 10 ± 5 | 10 ± 4 |
| salt thereof | | | | | | |
| HPMC | 10 ± 9 | 10 ± 8 | 10 ± 7 | 10 ± 6 | 10 ± 5 | 10 ± 4 |
| PEO M_{w} ≧ 500,000 | 50 ± 30 | 50 ± 27 | 50 ± 24 | 50 ± 21 | 50 ± 18 | 50 ± 15 |
| g/mol | | | | | | |
| Polyethylene glycol | 12 ± 11 | 12 ± 10 | 12 ± 9 | 12 ± 8 | 12 ± 7 | 12 ± 6 |
| α-tocopherol | 0.25 ± 0.24 | 0.25 ± 0.21 | 0.25 ± 0.18 | 0.25 ± 0.15 | 0.25 ± 0.12 | 0.25 ± 0.09 |
| Ingredient [wt.-%] | B³⁷ | B³⁸ | B³⁹ | B⁴⁰ | B⁴¹ | B⁴² |
| Opioid (A) | 19 ± 15 | 19 ± 13 | 19 ± 11 | 19 ± 9 | 19 ± 7 | 19 ± 5 |
| Anionic | 10 ± 9 | 10 ± 8 | 10 ± 7 | 10 ± 6 | 10 ± 5 | 10 ± 4 |
| polysaccharide (B) | | | | | | |
| Nonionic | 10 ± 9 | 10 ± 8 | 10 ± 7 | 10 ± 6 | 10 ± 5 | 10 ± 4 |
| polysaccharide | | | | | | |
| Polyalkylene oxide | 50 ± 30 | 50 ± 27 | 50 ± 24 | 50 ± 21 | 50 ± 18 | 50 ± 15 |
| (C) | | | | | | |
| Plasticizer | 12 ± 11 | 12 ± 10 | 12 ± 9 | 12 ± 8 | 12 ± 7 | 12 ± 6 |
| Antioxidant | 0.25 ± 0.24 | 0.25 ± 0.21 | 0.25 ± 0.18 | 0.25 ± 0.15 | 0.25 ± 0.12 | 0.25 ± 0.09 |
| Acid | 0.80 ± 0.75 | 0.80 ± 0.70 | 0.80 ± 0.65 | 0.80 ± 0.60 | 0.80 ± 0.55 | 0.80 ± 0.50 |
| Ingredient [wt.-%] | B⁴³ | B⁴⁴ | B⁴⁵ | B⁴⁶ | B⁴⁷ | B⁴⁸ |
| Opioid (A) | 19 ± 15 | 19 ± 13 | 19 ± 11 | 19 ± 9 | 19 ± 7 | 19 ± 5 |
| Croscarmellose or | 10 ± 9 | 10 ± 8 | 10 ± 7 | 10 ± 6 | 10 ± 5 | 10 ± 4 |
| salt thereof | | | | | | |
| HPMC | 10 ± 9 | 10 ± 8 | 10 ± 7 | 10 ± 6 | 10 ± 5 | 10 ± 4 |
| PEO M_{w} ≧ 500,000 | 50 ± 30 | 50 ± 27 | 50 ± 24 | 50 ± 21 | 50 ± 18 | 50 ± 15 |
| g/mol | | | | | | |
| Polyethylene glycol | 12 ± 11 | 12 ± 10 | 12 ± 9 | 12 ± 8 | 12 ± 7 | 12 ± 6 |
| α-tocopherol | 0.25 ± 0.24 | 0.25 ± 0.21 | 0.25 ± 0.18 | 0.25 ± 0.15 | 0.25 ± 0.12 | 0.25 ± 0.09 |
| Citric acid | 0.80 ± 0.75 | 0.80 ± 0.70 | 0.80 ± 0.65 | 0.80 ± 0.60 | 0.80 ± 0.55 | 0.80 ± 0.50 |

PEO = Polyethylene oxide

More particularly preferred compositions of the dosage form according to the invention are compiled as embodiments C¹ to C⁴⁸ in the tables here below (according to these embodiment, the dosage form according to the invention comprises the specified ingredients in the specified quantities but may additionally comprise further ingredients):

| Ingredient [wt.-%] | C¹ | C² | C³ | C⁴ | C⁵ | C⁶ |
|---|---|---|---|---|---|---|
| Oxymorphone or | 19 ± 15 | 19 ± 13 | 19 ± 11 | 19 ± 9 | 19 ± 7 | 19 ± 5 |
| salt thereof (A) | | | | | | |
| Anionic | 20 ± 15 | 20 ± 13 | 20 ± 11 | 20 ± 9 | 20 ± 7 | 20 ± 5 |
| polysaccharide (B) | | | | | | |
| Polyalkylene oxide | 50 ± 30 | 50 ± 27 | 50 ± 24 | 50 ± 21 | 50 ± 18 | 50 ± 15 |
| (C) | | | | | | |
| Ingredient [wt.-%] | C⁷ | C⁸ | C⁹ | C¹⁰ | C¹¹ | C¹² |
| Oxymorphone or | 19 ± 15 | 19 ± 13 | 19 ± 11 | 19 ± 9 | 19 ± 7 | 19 ± 5 |
| salt thereof (A) | | | | | | |
| Croscarmellose or | 20 ± 15 | 20 ± 13 | 20 ± 11 | 20 ± 9 | 20 ± 7 | 20 ± 5 |
| salt thereof | | | | | | |
| PEO M_{w} ≧ 500,000 | 50 ± 30 | 50 ± 27 | 50 ± 24 | 50 ± 21 | 50 ± 18 | 50 ± 15 |
| g/mol | | | | | | |
| Ingredient [wt.-%] | C¹³ | C¹⁴ | C¹⁵ | C¹⁶ | C¹⁷ | C¹⁸ |
| Oxymorphone or | 19 ± 15 | 19 ± 13 | 19 ± 11 | 19 ± 9 | 19 ± 7 | 19 ± 5 |
| salt thereof (A) | | | | | | |
| Anionic | 20 ± 15 | 20 ± 13 | 20 ± 11 | 20 ± 9 | 20 ± 7 | 20 ± 5 |
| polysaccharide (B) | | | | | | |
| Polyalkylene oxide | 50 ± 30 | 50 ± 27 | 50 ± 24 | 50 ± 21 | 50 ± 18 | 50 ± 15 |
| (C) | | | | | | |
| Plasticizer | 12 ± 11 | 12 ± 10 | 12 ± 9 | 12 ± 8 | 12 ± 7 | 12 ± 6 |
| Ingredient [wt.-%] | C¹⁹ | C²⁰ | C²¹ | C²² | C²³ | C²⁴ |
| Oxymorphone or | 19 ± 15 | 19 ± 13 | 19 ± 11 | 19 ± 9 | 19 ± 7 | 19 ± 5 |
| salt thereof (A) | | | | | | |
| Croscarmellose or | 20 ± 15 | 20 ± 13 | 20 ± 11 | 20 ± 9 | 20 ± 7 | 20 ± 5 |
| salt thereof | | | | | | |
| PEO M_{w} ≧ 500,000 | 50 ± 30 | 50 ± 27 | 50 ± 24 | 50 ± 21 | 50 ± 18 | 50 ± 15 |
| g/mol | | | | | | |
| Polyethylene glycol | 12 ± 11 | 12 ± 10 | 12 ± 9 | 12 ± 8 | 12 ± 7 | 12 ± 6 |
| Ingredient [wt.-%] | C²⁵ | C²⁶ | C²⁷ | C²⁸ | C²⁹ | C³⁰ |
| Oxymorphone or | 19 ± 15 | 19 ± 13 | 19 ± 11 | 19 ± 9 | 19 ± 7 | 19 ± 5 |
| salt thereof (A) | | | | | | |
| Anionic | 20 ± 15 | 20 ± 13 | 20 ± 11 | 20 ± 9 | 20 ± 7 | 20 ± 5 |
| polysaccharide (B) | | | | | | |
| Polyalkylene oxide | 50 ± 30 | 50 ± 27 | 50 ± 24 | 50 ± 21 | 50 ± 18 | 50 ± 15 |
| (C) | | | | | | |
| Plasticizer | 12 ± 11 | 12 ± 10 | 12 ± 9 | 12 ± 8 | 12 ± 7 | 12 ± 6 |
| Antioxidant | 0.25 ± 0.24 | 0.25 ± 0.21 | 0.25 ± 0.18 | 0.25 ± 0.15 | 0.25 ± 0.12 | 0.25 ± 0.09 |
| Ingredient [wt.-%] | C³¹ | C³² | C³³ | C³⁴ | C³⁵ | C³⁶ |
| Oxymorphone or | 19 ± 15 | 19 ± 13 | 19 ± 11 | 19 ± 9 | 19 ± 7 | 19 ± 5 |
| salt thereof (A) | | | | | | |
| Croscarmellose or | 20 ± 15 | 20 ± 13 | 20 ± 11 | 20 ± 9 | 20 ± 7 | 20 ± 5 |
| salt thereof | | | | | | |
| PEO M_{w} ≧ 500,000 | 50 ± 30 | 50 ± 27 | 50 ± 24 | 50 ± 21 | 50 ± 18 | 50 ± 15 |
| g/mol | | | | | | |
| Polyethylene glycol | 12 ± 11 | 12 ± 10 | 12 ± 9 | 12 ± 8 | 12 ± 7 | 12 ± 6 |
| α-tocopherol | 0.25 ± 0.24 | 0.25 ± 0.21 | 0.25 ± 0.18 | 0.25 ± 0.15 | 0.25 ± 0.12 | 0.25 ± 0.09 |
| Ingredient [wt.-%] | C³⁷ | C³⁸ | C³⁹ | C⁴⁰ | C⁴¹ | C⁴² |
| Oxymorphone or | 19 ± 15 | 19 ± 13 | 19 ± 11 | 19 ± 9 | 19 ± 7 | 19 ± 5 |
| salt thereof (A) | | | | | | |
| Anionic | 20 ± 15 | 20 ± 13 | 20 ± 11 | 20 ± 9 | 20 ± 7 | 20 ± 5 |
| polysaccharide (B) | | | | | | |
| Polyalkylene oxide | 50 ± 30 | 50 ± 27 | 50 ± 24 | 50 ± 21 | 50 ± 18 | 50 ± 15 |
| (C) | | | | | | |
| Plasticizer | 12 ± 11 | 12 ± 10 | 12 ± 9 | 12 ± 8 | 12 ± 7 | 12 ± 6 |
| Antioxidant | 0.25 ± 0.24 | 0.25 ± 0.21 | 0.25 ± 0.18 | 0.25 ± 0.15 | 0.25 ± 0.12 | 0.25 ± 0.09 |
| Acid | 0.80 ± 0.75 | 0.80 ± 0.70 | 0.80 ± 0.65 | 0.80 ± 0.60 | 0.80 ± 0.55 | 0.80 ± 0.50 |
| Ingredient [wt.-%] | C⁴³ | C⁴⁴ | C⁴⁵ | C⁴⁶ | C⁴⁷ | C⁴⁸ |
| Oxymorphone or | 19 ± 15 | 19 ± 13 | 19 ± 11 | 19 ± 9 | 19 ± 7 | 19 ± 5 |
| salt thereof (A) | | | | | | |
| Croscarmellose or | 20 ± 15 | 20 ± 13 | 20 ± 11 | 20 ± 9 | 20 ± 7 | 20 ± 5 |
| salt thereof | | | | | | |
| PEO M_{w} ≧ 500,000 | 50 ± 30 | 50 ± 27 | 50 ± 24 | 50 ± 21 | 50 ± 18 | 50 ± 15 |
| g/mol | | | | | | |
| Polyethylene glycol | 12 ± 11 | 12 ± 10 | 12 ± 9 | 12 ± 8 | 12 ± 7 | 12 ± 6 |
| α-tocopherol | 0.25 ± 0.24 | 0.25 ± 0.21 | 0.25 ± 0.18 | 0.25 ± 0.15 | 0.25 ± 0.12 | 0.25 ± 0.09 |
| Citric acid | 0.80 ± 0.75 | 0.80 ± 0.70 | 0.80 ± 0.65 | 0.80 ± 0.60 | 0.80 ± 0.55 | 0.80 ± 0.50 |

PEO = Polyethylene oxide

Preferably, the dosage form according to the invention is prepared by hot-melt extrusion.

Preferably, the pharmaceutical dosage form according to the invention is prepared by thermoforming, although also other methods of thermoforming may be used in order to manufacture the pharmaceutical dosage form according to the invention such as press-molding at elevated temperature or heating of tablets that were manufactured by conventional compression in a first step and then heated above the softening temperature of the polymer in the tablet in a second step to form hard tablets. In this regards, thermoforming means the forming or molding of a mass after the application of heat. In a preferred embodiment, the pharmaceutical dosage form is thermoformed by hot-melt extrusion.

In a preferred embodiment, the mixture of ingredients is heated and subsequently compressed under conditions (time, temperature and pressure) sufficient in order to achieve the desired mechanical properties, e.g. in terms of breaking strength and the like. This technique may be achieved e.g. by means of a tabletting tool which is either heated and/or which is filled with the heated mixture that is subsequently compressed without further supply of heat or with simultaneous additional supply of heat.

In another preferred embodiment, the mixture of ingredients is heated and simultaneously compressed under conditions (time, temperature and pressure) sufficient in order to achieve the desired mechanical properties, e.g. in terms of breaking strength and the like. This technique may be achieved e.g. by means of an extruder with one or more heating zones, wherein the mixture is heated and simultaneously subjected to extrusion forces finally resulting in a compression of the heated mixture.

In still another embodiment, the mixture of ingredients is compressed under ambient conditions at sufficient pressure and subsequently heated (cured) under conditions (time, temperature) sufficient in order to achieve the desired mechanical properties, e.g. in terms of breaking strength and the like. This technique may be achieved e.g. by means of a curing oven in which the compressed articles are cured for a sufficient time at a sufficient temperature, preferably without exerting any further pressure. Such process is further described e.g. in US 2009/0081290.

A particularly preferred process for the manufacture of the particles according to the invention involves hot-melt extrusion. In this process, the particles according to the invention are produced by thermoforming with the assistance of an extruder, preferably without there being any observable consequent discoloration of the extrudate.

In a preferred embodiment, the pharmaceutical dosage form is prepared by hot melt-extrusion, preferably by means of a twin-screw-extruder. Melt extrusion preferably provides a melt-extruded strand that is preferably cut into monoliths, which are then compressed and formed into tablets. In this regard, the term “tablets” is preferably not to be understood as dosage forms being made by compression of powder or granules (compressi) but rather, as shaped extrudates. Preferably, compression is achieved by means of a die and a punch, preferably from a monolithic mass obtained by melt extrusion. If obtained via melt extrusion, the compressing step is preferably carried out with a monolithic mass exhibiting ambient temperature, that is, a temperature in the range from 20 to 25° C. The strands obtained by way of extrusion can either be subjected to the compression step as such or can be cut prior to the compression step. This cutting can be performed by usual techniques, for example using rotating knives or compressed air. Alternatively, the shaping can take place as described in EP-A 240 906 by the extrudate being passed between two counter-rotating calender rolls and being shaped directly to tablets. It is of course also possible to subject the extruded strands to the compression step or to the cutting step when still warm, that is more or less immediately after the extrusion step. The extrusion is preferably carried out by means of a twin-screw extruder.

The pharmaceutical dosage form according to the invention may be produced by different processes, the particularly preferred of which are explained in greater detail below. Several suitable processes have already been described in the prior art. In this regard it can be referred to, e.g., WO 2005/016313, WO 2005/016314, WO 2005/063214, WO 2005/102286, WO 2006/002883, WO 2006/002884, WO 2006/002886, WO 2006/082097, and WO 2006/082099.

The present invention also relates to pharmaceutical dosage forms that are obtainable by any of the processes described here below.

In general, the process for the production of the pharmaceutical dosage form according to the invention preferably comprises the following steps:
(a) mixing all ingredients;
(b) optionally pre-forming the mixture obtained from step (a), preferably by applying heat and/or force to the mixture obtained from step (a), the quantity of heat supplied preferably not being sufficient to heat the polyalkylene oxide (C) up to its softening point;
(c) hardening the mixture by applying heat and force, and after the process decreasing heat and force, it being possible to supply the heat during and/or before the application of force and the quantity of heat supplied being sufficient to heat the polyalkylene oxide (C) at least up to its softening point;
(d) optionally singulating the hardened mixture;
(e) optionally shaping the pharmaceutical dosage form; and
(f) optionally providing a film coating.

Heat may be supplied directly, e.g. by contact or by means of hot gas such as hot air, or with the assistance of ultrasound; or is indirectly supplied by friction and/or shear. Force may be applied and/or the pharmaceutical dosage form may be shaped for example by direct tabletting or with the assistance of a suitable extruder, particularly by means of a screw extruder equipped with two screws (twin-screw-extruder) or by means of a planetary gear extruder.

The final shape of the pharmaceutical dosage form may either be provided during the hardening of the mixture by applying heat and force (step (c)) or in a subsequent step (step (e)). In both cases, the mixture of all components is preferably in the plastified state, i.e. preferably, shaping is performed at a temperature at least above the softening point of the polyalkylene oxide (C). However, extrusion at lower temperatures, e.g. ambient temperature, is also possible and may be preferred.

Shaping can be performed, e.g., by means of a tabletting press comprising die and punches of appropriate shape.

A particularly preferred process for the manufacture of the pharmaceutical dosage form of the invention involves hot-melt extrusion. In this process, the pharmaceutical dosage form according to the invention is produced by thermoforming with the assistance of an extruder, preferably without there being any observable consequent discoloration of the extrudate.

This process is characterized in that
a) all components are mixed,
b) the resultant mixture is heated in the extruder at least up to the softening point of the polyalkylene oxide (C) and extruded through the outlet orifice of the extruder by application of force,
c) the still plastic extrudate is singulated and formed into the pharmaceutical dosage form or
d) the cooled and optionally reheated singulated extrudate is formed into the pharmaceutical dosage form.

Mixing of the components according to process step a) may also proceed in the extruder.

The components may also be mixed in a mixer known to the person skilled in the art. The mixer may, for example, be a roll mixer, shaking mixer, shear mixer or compulsory mixer.

The, preferably molten, mixture which has been heated in the extruder at least up to the softening point of polyalkylene oxide (C) is extruded from the extruder through a die with at least one bore.

The process according to the invention requires the use of suitable extruders, preferably screw extruders. Screw extruders which are equipped with two screws (twin-screw-extruders) are particularly preferred.

The extrusion is preferably performed so that the expansion of the strand due to extrusion is not more than 30%, i.e. that when using a die with a bore having a diameter of e.g. 6 mm, the extruded strand should have a diameter of not more than 8 mm. More preferably, the expansion of the strand is not more than 25%, still more preferably not more than 20%, most preferably not more than 15% and in particular not more than 10%.

Preferably, extrusion is performed in the absence of water, i.e., no water is added. However, traces of water (e.g., caused by atmospheric humidity) may be present.

The extruder preferably comprises at least two temperature zones, with heating of the mixture at least up to the softening point of the polyalkylene oxide (C) preceding in the first zone, which is downstream from a feed zone and optionally mixing zone. The throughput of the mixture is preferably from 1.0 kg to 15 kg/hour. In a preferred embodiment, the throughput is from 1 to 3.5 kg/hour. In another preferred embodiment, the throughput is from 4 to 15 kg/hour.

In a preferred embodiment, the die head pressure is within the range of from 25 to 100 bar. The die head pressure can be adjusted inter alia by die geometry, temperature profile and extrusion speed.

The die geometry or the geometry of the bores is freely selectable. The die or the bores may accordingly exhibit a round, oblong or oval cross-section, wherein the round cross-section preferably has a diameter of 0.1 mm to 15 mm and the oblong cross-section preferably has a maximum lengthwise extension of 21 mm and a crosswise extension of 10 mm. Preferably, the die or the bores have a round cross-section. The casing of the extruder used according to the invention may be heated or cooled. The corresponding temperature control, i.e. heating or cooling, is so arranged that the mixture to be extruded exhibits at least an average temperature (product temperature) corresponding to the softening temperature of the polyalkylene oxide (C) and does not rise above a temperature at which the opioid (A) to be processed may be damaged. Preferably, the temperature of the mixture to be extruded is adjusted to below 180° C., preferably below 150° C., but at least to the softening temperature of polyalkylene oxide (C). Typical extrusion temperatures are 120° C. and 130° C.

In a preferred embodiment, the extruder torque is within the range of from 30 to 95%. Extruder torque can be adjusted inter alia by die geometry, temperature profile and extrusion speed.

After extrusion of the molten mixture and optional cooling of the extruded strand or extruded strands, the extrudates are preferably singulated. This singulation may preferably be performed by cutting up the extrudates by means of revolving or rotating knives, water jet cutters, wires, blades or with the assistance of laser cutters.

Preferably, intermediate or final storage of the optionally singulated extrudate or the final shape of the pharmaceutical dosage form according to the invention is performed under oxygen-free atmosphere which may be achieved, e.g., by means of oxygen-scavengers.

The singulated extrudate may be press-formed into tablets in order to impart the final shape to the pharmaceutical dosage form.

The application of force in the extruder onto the at least plasticized mixture is adjusted by controlling the rotational speed of the conveying device in the extruder and the geometry thereof and by dimensioning the outlet orifice in such a manner that the pressure necessary for extruding the plasticized mixture is built up in the extruder, preferably immediately prior to extrusion. The extrusion parameters which, for each particular composition, are necessary to give rise to a pharmaceutical dosage form with desired mechanical properties, may be established by simple preliminary testing.

For example but not limiting, extrusion may be performed by means of a twin-screw-extruder type ZSE 18 or ZSE27 (Leistritz, Nurnberg, Germany), screw diameters of 18 or 27 mm. Screws having eccentric ends may be used. A heatable die with a round bore having a diameter of 7, 8, or 9 mm may be used. The extrusion parameters may be adjusted e.g. to the following values: rotational speed of the screws: 120 Upm; delivery rate 2 kg/h for a ZSE 18 or 8 kg/h for a ZSE27; product temperature: in front of die 125° C. and behind die 135° C.; and jacket temperature: 110° C.

Preferably, extrusion is performed by means of twin-screw-extruders or planetary-gear-extruders, twin-screw extruders (co-rotating or contra-rotating) being particularly preferred.

The process for the preparation of the pharmaceutical dosage form according to the invention is preferably performed continuously. Preferably, the process involves the extrusion of a homogeneous mixture of all components. It is particularly advantageous if the thus obtained intermediate, e.g. the strand obtained by extrusion, exhibits uniform properties. Particularly desirable are uniform density, uniform distribution of the active ingredient, uniform mechanical properties, uniform porosity, uniform appearance of the surface, etc. Only under these circumstances the uniformity of the pharmacological properties, such as the stability of the release profile, may be ensured and the amount of rejects can be kept low.

A further aspect of the invention relates to the use of an opioid (A) for the manufacture of the pharmaceutical dosage form as described above for the treatment of pain. A further aspect of the invention relates to the use of a pharmaceutical dosage form as described above for avoiding or hindering the abuse of the opioid (A) contained therein. A further aspect of the invention relates to the use of a pharmaceutical dosage form as described above for avoiding or hindering the abuse and/or the intentional or unintentional overdose of the opioid (A) contained therein. In this regard, the invention also relates to the use of an opioid (A) as described above and/or an anionic polysaccharide (B) as described above and/or a polyalkylene oxide (C) as described above for the manufacture of the pharmaceutical dosage form according to the invention for the prophylaxis and/or the treatment of a disorder, thereby preventing an abuse and/or the intentional or unintentional of the opioid (A), particularly due to comminution of the pharmaceutical dosage form by mechanical action and/or solvent extraction.

Further, the invention relates to a method for the prophylaxis and/or the treatment of a disorder comprising the administration of the pharmaceutical dosage form according to the invention, thereby preventing an overdose of the opioid (A), particularly due to comminution of the pharmaceutical dosage form by mechanical action and/or solvent extraction. Preferably, the mechanical action is selected from the group consisting of chewing, grinding in a mortar, pounding, and using apparatuses for pulverizing conventional pharmaceutical dosage forms.

The following examples further illustrate the invention but are not to be construed as limiting its scope:

Cut rods having essentially the same composition (#1, #2, #3, #4, #5, #6 and #7, respectively) but containing different opioids (Hydrocodone, Oxycodone, Oxymorphone, and Tapentadol, respectively) were manufactured by hot melt extrusion:

| | comparative | | | inventive | | comparative | |
|---|---|---|---|---|---|---|---|
| Ingredient [wt.-%] | #1 | #2 | #3 | #4 | #5 | #6 | #7 |
| Opioid | 18.60 | 18.60 | 18.60 | 18.60 | 18.60 | 18.60 | 18.60 |
| PEO 7 Mio. | 56.80 | 48.72 | 48.73 | 48.73 | 48.73 | 48.73 | 48.73 |
| PEG 6000 | 13.52 | 11.60 | 11.60 | 11.60 | 11.60 | 11.60 | 11.60 |
| Citric acid | 0.84 | 0.84 | 0.84 | 0.84 | 0.84 | 0.84 | 0.84 |
| α-Tocopherol | 0.23 | 0.23 | 0.23 | 0.23 | 0.23 | 0.23 | 0.23 |
| HPMC 100000 mPas | 10.00 | 10.00 | — | 10.00 | — | 10.00 | 10.00 |
| Xanthan gum | — | 10.00 | 20.00 | — | — | — | — |
| Crosscarmellose Sodium | — | — | — | 10.00 | 20.00 | — | — |
| Starch 1500 | — | — | — | — | — | 10.00 | — |
| Carbopol 71 G | — | — | — | — | — | — | 10.00 |

The cut rods #4 and #5 contained croscarmallose sodium and thus are in accordance with the present invention. The cut rods #1, #2, #3, #6 and #7 are not in accordance with the invention, i.e. comparative.

Hydrocodone was employed in form of its tartrate salt (Hydrocodone hydrogentartrate 2.5 hydrate), whereas Oxycodone, Oxymorphone and Tapentadol were each employed in form of their hydrochloride salts.

Each cut rod had a total weight of 215 mg.

Polyethylene oxide, α-tocopherol, opioid and all other excipients were weighted and sieved. The powder was mixed and dosed gravimetrically to an extruder. Hot-melt extrusion was performed by means of a twin screw extruder of type ZSE 18 (Leistritz, Nurnberg, Germany) that was equipped with a heatable round die having a diameter of 7 mm. The hot extrudate was cooled on a conveyor belt and the cooled extrusion strand was comminuted to cut rods.

The breaking strength of the cut rods was measured by means of a Sotax® HT100 at a constant speed of 120 mm/min and/or a Zwick Z 2.5 at a constant speed of 10 mm/min. A cut rod was regarded as failing the breaking strength test when during the measurement the force dropped below the threshold value of 25% of the maximum force that was observed during the measurement, regardless of whether the cut rod was fractured into separate pieces or not. All values are given as mean of 3 measurements (Zwick; n=3) or as a mean of 10 measurements (Sotax, n=10).

All cut rods had a breaking strength of more than 1000 N.

The in vitro release profile of the opioid from the cut rods was measured in 600 ml of artificial gastric juice (pH 6.8) at temperature of 37° C. with sinker (type 4). The rotation speed of the paddle was adjusted to 75/min. The opioid was detected by means of a spectrometric measurement.

The in vitro release profiles are shown in FIGS. 1 to 4 demonstrating that all cut rods provided prolonged release of opioids (A). FIG. 1: Hydrocodone; FIG. 2: Oxymorphone; FIG. 3: Oxycodone; FIG. 4: Tapentadol.

The extractability of Hydrocodone, Oxycodone, Oxymorphone and Tapentadol from the various dosage forms (cut rods) was tested.

The results of the test with 5 ml water, boiled for 5 min and subsequently filtered per G21 (n=3) are shown in FIG. 5.

The results of the test with 30 ml medium, 30 min (n=3) are shown in FIGS. 6 to 9. FIG. 6 shows the results of an extraction test for Hydrocodone with 30 ml medium, 30 min. FIG. 7 shows the results of an extraction test for Oxymorphone with 30 ml medium, 30 min. FIG. 8 shows the results of an extraction test for Oxycodone with 30 ml medium, 30 min. FIG. 9 shows the results of an extraction test for Tapentadol with 30 ml medium, 30 min.

The results for the individual pharmacologically active ingredients are shown in the tables here below. The worst dosage forms are mentioned to the left whereas the best dosage forms are mentioned to the right.

Hydrocodone (n=3):

| | #4 | #7 | #2 | | | | #6 |
|---|---|---|---|---|---|---|---|
| | 10% | 10% | 10% | | | | 10% |
| | HPMC + | HPMC + | HPMC + | #1 | #5 | #3 | HPMC + |
| | 10% Cros- | 10% | 10% | 10% | 20% Cros- | 20% | 10% |
| | carmellose | Carbopol | Xanthan | HPMC | carmellose | Xanthan | Starch |
| mean | 32.62 | 30.29 | 30.16 | 28.12 | 26.91 | 26.27 | 25.59 |
| SD | 2.36 | 4.61 | 3.44 | 7.07 | 0.74 | 6.04 | 4.95 |

→ improved resistance against solvent extraction →

Oxycodone (n=3):

| | #7 | | | #2 | #4 | | #6 |
|---|---|---|---|---|---|---|---|
| | 10% | | | 10% | 10% | | 10% |
| | HPMC. | #1 | #3 | HPMC + | HPMC + | #5 | HPMC. |
| | 10% | 10% | 20% | 10% | 10% | 20% | 10% |
| | Carbopol | HPMC | Xanthan | Xanthan | Croscarmellose | Croscarmellose | Starch |
| mean | 25.13 | 21.56 | 19.63 | 19.60 | 15.27 | 13.15 | 11.02 |
| SD | 1.30 | 3.03 | 2.68 | 0.99 | 2.12 | 2.74 | 0.49 |

→ improved resistance against solvent extraction →

Oxymorphone (n=3):

| | | #2 | #4 | #7 | | #6 | |
|---|---|---|---|---|---|---|---|
| | | 10% | 10% | 10% | | 10% | |
| | #1 | HPMC + | HPMC + | HPMC + | #3 | HPMC + | #5 |
| | 10% | 10% | 10% | 10% | 20% | 10% | 20% |
| | HPMC | Xanthan | Croscarmellose | Carbopol | Xanthan | Starch | Croscarmellose |
| mean | 34.36 | 31.49 | 28.65 | 22.00 | 18.99 | 18.30 | 6.23 |
| SD | 8.95 | 17.45 | 10.98 | 13.55 | 3.09 | 11.48 | 0.58 |

→ improved resistance against solvent extraction →

Tapentadol (n=3):

| | | #7 | #2 | | #4 | | #6 |
|---|---|---|---|---|---|---|---|
| | | 10% | 10% | | 10% | | 10% |
| | #3 | HPMC + | HPMC + | #1 | HPMC + | #5 | HPMC + |
| | 20% | 10% | 10% | 10% | 10% | 20% | 10% |
| | Xanthan | Carbopol | Xanthan | HPMC | Croscarmellose | Croscarmellose | Starch |
| mean | 19.52 | 18.62 | 18.42 | 16.40 | 13.26 | 13.45 | 10.04 |
| SD | 2.38 | 1.71 | 1.16 | 4.42 | 1.47 | 2.48 | 1.86 |

→ improved resistance against solvent extraction →

These results are also shown in FIG. 10.

The relative improvement of the resistance against solvent extraction compared to #1 (10% HPMC) is quantified in the tables here below:

Hydrocodone:

| | | | inventive | | | |
|---|---|---|---|---|---|---|
| | comparative | | #4 | | comparative | |
| | #2 | #3 | HPMC + | #5 | #6 | #7 |
| | HPMC + | Xan- | Croscar- | Croscar- | HPMC + | HPMC + |
| | Xanthan | than | mellose | mellose | Starch | Carbopol |
| water, | +1.77 | −0.41 | −1.40 | −2.99 | +4.93 | +1.90 |
| RT | | | | | | |
| water, | −2.40 | −4.44 | −4.37 | −6.87 | +0.56 | −6.75 |
| boiled | | | | | | |
| ethanol | +1.12 | +1.02 | −0.33 | −0.31 | +1.55 | +1.53 |

Oxycodone:

| | | | inventive | | | |
|---|---|---|---|---|---|---|
| | comparative | | #4 | | comparative | |
| | #2 | #3 | HPMC + | #5 | #6 | #7 |
| | HPMC + | Xan- | Croscar- | Croscar- | HPMC + | HPMC + |
| | Xanthan | than | mellose | mellose | Starch | Carbopol |
| water, | +2.66 | +0.81 | −3.65 | −7.56 | +3.67 | −0.75 |
| RT | | | | | | |
| water, | −5.07 | −4.65 | −11.72 | −16.46 | −1.38 | −3.49 |
| boiled | | | | | | |
| ethanol | +3.98 | +0.98 | −1.24 | −2.84 | +2.33 | +4.90 |

Oxymorphone:

| | | | inventive | | | |
|---|---|---|---|---|---|---|
| | comparative | | #4 | | comparative | |
| | #2 | #3 | HPMC + | #5 | #6 | #7 |
| | HPMC + | Xan- | Croscar- | Croscar- | HPMC + | HPMC + |
| | Xanthan | than | mellose | mellose | Starch | Carbopol |
| water, | −0.26 | +0.12 | −1.47 | −5.03 | +1.19 | −0.82 |
| RT | | | | | | |
| water, | −2.04 | −4.41 | −4.26 | −10.67 | −0.62 | −3.60 |
| boiled | | | | | | |
| ethanol | +1.11 | +0.42 | −2.35 | −2.68 | +0.40 | −0.27 |

Tapentadol:

| | | | inventive | | | |
|---|---|---|---|---|---|---|
| | comparative | | #4 | | comparative | |
| | #2 | #3 | HPMC + | #5 | #6 | #7 |
| | HPMC + | Xan- | Croscar- | Croscar- | HPMC + | HPMC + |
| | Xanthan | than | mellose | mellose | Starch | Carbopol |
| water, | +0.77 | +1.29 | −1.31 | −2.25 | +0.90 | −0.55 |
| RT | | | | | | |
| water, | +1.48 | −2.80 | −1.61 | −6.58 | +5.41 | −2.22 |
| boiled | | | | | | |
| ethanol | −1.15 | −1.72 | −2.14 | −3.97 | −1.35 | −1.60 |

RT = room temperature

As demonstrated by the above comparative data, the pharmaceutical dosage forms according to the invention provide a substantially improved resistance against extraction with various solvents under various conditions and still provide prolonged release of the opioids and increased breaking strength, i.e. resistance to crushing.

1. A pharmaceutical dosage form having a breaking strength of at least 300 N, said dosage form comprising
an opioid (A) selected from the group consisting of Oxymorphone, Oxycodone, Tapentadol, Hydromorphone, Hydrocodone, Morphine, and the physiologically acceptable salts thereof; wherein the weight content of the opioid (A) is within the range of from 5.0 to 35 wt.-%, based on the total weight of the pharmaceutical dosage form;
an anionic polysaccharide (B) selected from the group consisting of croscarmellose, carmellose, crosslinked carboxymethyl starch, carboxymethyl starch, and the physiologically acceptable salts thereof; wherein the weight content of the anionic polysaccharide (B) is within the range of from 5.0 to 35 wt.-%, based on the total weight of the pharmaceutical dosage form; and
a polyalkylene oxide (C) having a weight average molecular weight of at least 200,000 g/mol; wherein the weight content of the polyalkylene oxide (C) is within the range of from 20 to 80 wt.-%, based on the total weight of the pharmaceutical dosage form;
wherein the opioid (A) is present in a controlled-release matrix comprising the anionic polysaccharide (B) and the polyalkylene oxide (C).

2. The dosage form according to claim 1, wherein the anionic polysaccharide (B) is selected from the group consisting of croscarmellose, carmellose, crosslinked carboxymethyl starch, carboxymethyl starch, and the physiologically acceptable salts thereof.

3. (canceled)

4. (canceled)

5. (canceled)

6. (canceled)

7. (canceled)

8. (canceled)

9. The dosage form according to claim 1, wherein the opioid (A) is Oxymorphone or a physiologically acceptable salt thereof.

10. The dosage form according to claim 1, wherein the opioid (A) is Oxycodone or a physiologically acceptable salt thereof.

11. The dosage form according to claim 1, wherein the opioid (A) is Tapentadol or a physiologically acceptable salt thereof.

12. The dosage form according to claim 1, wherein the opioid (A) is Hydromorphone or a physiologically acceptable salt thereof.

13. The dosage form according to claim 1, wherein the opioid (A) is Hydrocodone or a physiologically acceptable salt thereof.

14. The dosage form according to claim 1, wherein the opioid (A) is Morphine or a physiologically acceptable salt thereof.

15. The dosage form according to claim 1, wherein the weight content of the opioid (A) is within a range selected from the group consisting of 20±10 wt.-%, 19±15 wt.-%, 19±13 wt.-%, 19±11 wt.-%, 19±9 wt.-%, 19±7 wt.-%, and 19±5 wt.-%, wherein all wt.-%'s are based on a total weight of the dosage form.

16. (canceled)

17. (canceled)

18. (canceled)

19. (canceled)

20. (canceled)

21. (canceled)

22. The dosage form according to claim 1, wherein the weight content of the anionic polysaccharide (B) is within a range selected from the group consisting of 20±15 wt.-%, 20±13 wt.-%, 20±11 wt.-%, 20±10 wt.-%, 20±9 wt.-%, 20±7 wt.-%, and 20±5 wt.-%, wherein all wt.-%'s are based on the total weight of the dosage form.

23. (canceled)

24. (canceled)

25. (canceled)

26. (canceled)

27. (canceled)

28. (canceled)

29. The dosage form according to claim 1, wherein the weight content of the polyalkylene oxide (C) is within a range selected from the group consisting of 50±30 wt.-%, 50±27 wt.-%, 50±24 wt.-%, 50±21 wt.-%, 50±20 wt.-%, 50±18 wt.-%, and 50±15 wt.-%, wherein all wt.-%'s are based on the total weight of the dosage form.

30. (canceled)

31. (canceled)

32. (canceled)

33. (canceled)

34. (canceled)

35. (canceled)

36. The dosage form according to claim 1, wherein the relative weight ratio of the polyalkylene oxide (C) to the anionic polysaccharide (B) is within a range selected from the group consisting of from 8:1 to 1:1, 7:1 to 1:1, 6:1 to 1.5:1, 5:1 to 1.5:1, 4:1 to 2:1 and 3:1 to 2:1.

37. (canceled)

38. (canceled)

39. (canceled)

40. (canceled)

41. (canceled)

42. The dosage form according to claim 1, wherein the relative weight ratio of the polyalkylene oxide (C) to the opioid (A) is within a range selected from the group consisting of from 8:1 to 1:1, 7:1 to 1:1, 6:1 to 1.5:1, 5:1 to 1.5:1, 4:1 to 2:1 and 3:1 to 2:1.

43. (canceled)

44. (canceled)

45. (canceled)

46. (canceled)

47. (canceled)

48. The dosage form according to claim 1, wherein the relative weight ratio of the opioid (A) to the anionic polysaccharide (B) is within a range selected from the group consisting of from 4:1 to 1:4, 3.5:1 to 1:3.5, 3:1 to 1:3, 2.5:1 to 1:2.5, 2:1 to 1:2 and 1.5:1 to 1:1.5.

49. (canceled)

50. (canceled)

51. (canceled)

52. (canceled)

53. (canceled)

54. The dosage form according to claim 1, which provides a release of the opioid (A)
after 1 hour of at most 60%, or at most 40%, or at most 30%, or at most 20%, or at most 17%;
after 2 hours at most 80%, or at most 60%, or at most 50%, or at most 40%, or at most 32%;
after 3 hours at most 85%, or at most 65%, or at most 55%, or at most 48%, or at most 42%;
after 4 hours at most 90%, or at most 75%, or at most 65%, or at most 55%, or at most 49%;
after 7 hours at most 95%, or at most 85%, or at most 80%, or at most 70%, or at most 68%;
after 10 hours at most 99%, or at most 90%, or at most 88%, or at most 83%, or at most 80%; and/or
after 13 hours at most 99%, or at most 95%, or at most 93%, or at most 91%, or at most 89%.

55. The dosage form according to claim 1 which has released under in vitro conditions:
after 1 h at most 40 wt.-%,
after 2 h at most 55 wt.-%,
after 3 h at most 70 wt.-%, and
after 4 h at most 85 wt.-%
of the total content of the opioid (A) that was originally contained in the dosage form.

56. The dosage form according to claim 1, wherein the polyalkylene oxide (C) is a polyethylene oxide.

57. The dosage form according to claim 1, wherein the polyalkylene oxide (C) has a weight average molecular weight of at least 0.5 million g/mol.

58. (canceled)

59. The dosage form according to claim 1, which additionally comprises a non-ionic polysaccharide selected from the group consisting of methylcellulose, ethylcellulose, propylcellulose, hydroxyethylcellulose, hydroxypropylcellulose and hydroxypropylmethylcellulose.

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. A method of treating pain comprising administering to a patient in need thereof a dosage form according to claim 1.
